(19) United States
(12) Patent Application Publication
(10) Pub. No.: US 2019/0314285 A1
(43) Pub. Date: Oct. 17, 2019

(54) DOSAGE FORMS COMPRISING ABIRATERONE ACETATE

(71) Applicant: Janssen Pharmaceutica NV, Beerse (BE)

(72) Inventors: Jurgen Mensch, Laakdal (BE); Urbain Alfons C. Delaet, Beerse (BE); Philip Erna H. Heyns, Vosselaar (BE); Claire Elisabeth Balmain, Beerse (BE)

(21) Appl. No.: 16/380,138

(22) Filed: Apr. 10, 2019

Related U.S. Application Data
(60) Provisional application No. 62/657,499, filed on Apr. 13, 2018.

(51) Int. Cl. A61K 9/28; A61K 31/58; A61P 35/00; A61K 9/00; A61K 9/20

(52) U.S. Cl. CPC A61K 9/28; A61K 31/58; A61P 35/00; A61K 9/2009; A61K 9/2054; A61K 9/2027; A61K 9/2013; A61K 9/0053

(57) ABSTRACT

Disclosed are solid oral dosage forms comprising about 500 mg of abiraterone acetate; and a film coating that is positioned on an outer surface of said dosage form, wherein the dosage forms are bioequivalent to 250 mg ZYTIGA® abiraterone acetate dosage forms when administered orally on an equivalent dose basis. Also disclosed are approved drug products comprising 500 mg abiraterone acetate, as are methods of treatment, of reducing pill burden, and of providing pharmaceutical regimen that is bioequivalent to 250 mg ZYTIGA® abiraterone acetate dosage forms when administered orally on an equivalent dose basis.

### TECHNICAL FIELD

The present disclosure relates to oral dosage forms for the treatment of prostate cancer.

### BACKGROUND

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens such as testosterone promote prostate cancer growth. At its early stages, localized prostate cancer is often curable with local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts).

For many years, the established standard of care for men with malignant castration-resistant prostate cancer (mCRPC) was docetaxel chemotherapy. More recently, androgen receptor (AR)-targeted agents such as enzalutamide (XTANDI®) have improved time to progression and survival rates, when indirectly compared to docetaxel. However, there remains a subset of patients who either do not respond initially, or become refractory (or resistant) to these treatments. No approved therapeutic options are available for such patients. Platinum-based chemotherapy has been tested in a number of clinical studies in molecularly unselected prostate cancer patients with limited results and significant toxicities.

Recently, the PARP inhibitor, olaparib, was investigated in a Phase 2 study to assess efficacy and safety in patients with mCRPC post-chemotherapy and AR-targeted agents. Genetic sequencing identified homozygous deletions, deleterious mutations, or both in DNA-repair genes, including, but not limited to BRCA-1/2, ATM, Fanconi anemia genes, and CHEK2 in tumor samples. Sixteen of 49 patients had a response (33%; 95% confidence interval [CI]: 20%, 48%). Response was defined as one or more of the following: objective response, circulating tumor cell (CTC) conversion, or prostate specific antigen (PSA) decline ≤50%. Of these 16 patients, 14 (88%) had a response to olaparib and were biomarker-positive for anomalies in DNA-repair genes, including all 7 patients with BRCA-2 loss (4 with bi-allelic somatic loss, and 3 with germline mutations) and 4 of 5 patients with ATM aberrations. Conversely, only 2 of 33 biomarker-negative patient tumors (6%) had a response. Radiographic progression-free survival (rPFS) was significantly longer in the biomarker-positive group than in the biomarker-negative group (median: 9.8 versus 2.7 months, respectively). Overall survival (OS) was also prolonged in the biomarker-positive group versus the biomarker-negative group (median: 13.8 months versus 7.5 months, respectively).

However, a need remains for safe and efficacious therapies against prostate cancer, including hormone sensitive and castration-resistant prostate cancers.

### SUMMARY

Provided herein are solid oral dosage forms comprising about 500 mg of abiraterone acetate and having a dissolution profile characterized by one or more of features (a)-(f):

(a) about 43% of said dosage form dissolves after five minutes;

(b) about 71% of said dosage form dissolves after 10 minutes;

(c) about 88% of said dosage form dissolves after 20 minutes;

(d) about 94% of said dosage form dissolves after 30 minutes;

(e) about 98% of said dosage form dissolves after 45 minutes; and,

(f) about 99% of said dosage form dissolves after 60 minutes,

when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

Also provided herein are solid oral dosage forms comprising about 500 mg of abiraterone acetate and having a dissolution profile characterized by one or more of features (a)-(f):

(a) about 31% of said dosage form dissolves after five minutes;

(b) about 65% of said dosage form dissolves after 10 minutes;

(c) about 94% of said dosage form dissolves after 20 minutes;

(d) about 98% of said dosage form dissolves after 30 minutes;

(e) about 99% of said dosage form dissolves after 45 minutes; and,

(f) about 99% of said dosage form dissolves after 60 minutes,

when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

Also disclosed are solid oral dosage forms comprising about 500 mg of abiraterone acetate; and a film coating that is positioned on an outer surface of said dosage form, wherein said dosage forms are bioequivalent, when administered orally on an equivalent dose basis, to 250 mg ZYTIGA® abiraterone acetate dosage forms.

The present disclosure also provides methods of reducing pill burden on a subject in need of an abiraterone acetate pharmaceutical regimen comprising orally administering to the subject two dosage forms according to the present disclosure at substantially the same time.

Also provided are methods of treating a subject using an abiraterone acetate pharmaceutical regimen that is bioequivalent to 250 mg ZYTIGA® abiraterone acetate dosage forms when administered orally on an equivalent dose basis, comprising orally administering a dosage form according to the present disclosure.

The present disclosure also provides methods of treating a subject who has prostate cancer comprising orally administering to said subject a dosage form according to the present disclosure.

Also provided are methods of selling a drug product comprising abiraterone acetate, said method comprising selling such drug product, wherein a drug product label for a reference listed drug for such drug product includes instructions for treating non-metastatic castration resistant prostate cancer.

Also disclosed are methods of offering for sale a drug product comprising abiraterone acetate, said method comprising offering for sale such drug product, wherein a drug product label for a reference listed drug for such drug product includes instructions for treating non-metastatic castration resistant prostate cancer.

The present disclosure also provides methods of selling a drug product comprising abiraterone acetate, said method comprising selling such drug product, wherein the drug product label for a reference listed drug for such drug product comprises metastasis free survival data.

Also disclosed are methods of offering for sale a drug product comprising abiraterone acetate, said method comprising offering for sale such drug product, wherein the drug product label for a reference listed drug for such drug product comprises metastasis free survival data.

The present disclosure also provides an approved drug product comprising 500 mg abiraterone acetate.

### BRIEF DESCRIPTION OF THE DRAWINGS

FIGS. 1A and 1B illustrate Arithmetic Mean (+SD) Plasma Concentration-Time Profiles of Abiraterone (Study 212082PCR1010).

FIGS. 2A and 2B illustrate Arithmetic Mean (+SD) Plasma Concentration-Time Profiles of Abiraterone (Study 212082PCR1007).

FIGS. 3A and 3B provide results of a cross-study Comparison of Abiraterone Cₘₐₓ (3A) and AUC_{∞} (3B) Following Oral Administration of 1,000 mg of Abiraterone Acetate as 4×250-mg (G004) and 2×500-mg (G023) FC (film coated) Tablets (Studies 212082PCR1010 and 212082PCR1007).

FIGS. 4A-4G illustrate the results of an assessment of release and stability by the inventive 500 mg film-coated tablet dosage forms at specified time points (5, 10, 15, 20, 30, 45, and 60 minutes, respectively).

FIG. 5 illustrates individual dissolution profiles of batch 4207 of the 500-mg FC tablets (G023) obtained using the chosen dissolution method.

FIG. 6 illustrates average dissolution profiles and SD of batch 4207 of the 500-mg FC tablets (G023) as measured with a paddle apparatus at different rotation speeds (50, 60, and 75 rpm) using 900 mL phosphate buffer of pH 4.5 containing 0.25% of SLS at 37° C. (N=6).

FIG. 7 shows average dissolution profiles and SD of batch 4207 stored under stress conditions, tested with the selected dissolution method (N=6).

FIG. 8 shows average dissolution profiles and SD of G023) FC (film coated) Tablets batch JNJ-212082-n006-00399 measured with a paddle apparatus at 75 rpm using 900 mL dissolution media with different pH containing 0.25% SLS at 37° C. (N=6).

FIG. 9 shows average dissolution profiles and SD of batch JNJ-212082-n006-00399 measured with a paddle apparatus at 75 rpm using 900 mL Phosphate Buffer pH 4.5 containing different % SLS at 37° C.

### DETAILED DESCRIPTION OF ILLUSTRATIVE EMBODIMENTS

The present inventions may be understood more readily by reference to the following detailed description taken in connection with the accompanying examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “an ingredient” is a reference to one or more of such ingredients and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

The present disclosure relates, inter alia, to dosage forms comprising abiraterone acetate and methods for treating prostate cancer comprising administering such dosage forms to a subject in need thereof.

Abiraterone acetate (available under the trade name ZYTIGA®), illustrated below, is a 17a-hydroxylase/C17,20-lyase (CYP17) inhibitor that blocks androgen biosynthesis in the testes, adrenal gland, and prostate tumor.

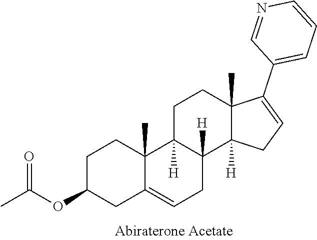

After oral administration, abiraterone acetate, the prodrug form in the commercial preparation, is converted into the active form, abiraterone. This conversion is likely to be esterase-mediated and not CYP-mediated. Administration with food increases absorption of the drug and thus has the potential to result in increased and highly variable exposures; the drug should be consumed on an empty stomach at least one hour before or two hours after food. The drug is highly protein bound (>99%), and is metabolised in the liver by CYP3A4 and SULT2A1 to inactive metabolites.

Abiraterone, the active metabolite of abiraterone acetate, inhibits CYP17A1, which manifests as two enzymes, 17α-hydroxylase (IC₅₀=2.5 nM) and 17,20-lyase (IC50=15 nM) (six-fold more selective for inhibition of 17α-hydroxylase over 17,20-lyase)[12] that are expressed in testicular, adrenal, and prostatic tumor tissues. CYP17 catalyzes two sequential reactions: (1) the conversion of pregnenolone and progesterone to their 17α-hydroxy derivatives by its 17α-hydroxylase activity, and (2) the subsequent formation of dehydroepiandrosterone (DHEA) and androstenedione, respectively, by its 17,20-lyase activity. DHEA and androstenedione are androgens and precursors of testosterone. Inhibition of CYP17 activity by abiraterone thus decreases circulating levels of androgens such as DHEA, testosterone, and dihydrotestosterone (DHT).

Abiraterone also acts as a partial antagonist of the androgen receptor (AR), and as an inhibitor of the enzymes 3β-hydroxysteroid dehydrogenase (3β-HSD), CYP11B1 (steroid 11β-hydroxylase), and other CYP450s (e.g., CYP1A2, CYP2C9, and CYP3A4). In addition to abiraterone itself, part of the activity of the drug has been found to be due to a more potent active metabolite, Δ⁴-abiraterone (D4A), which is formed from abiraterone by 3β-HSD. D4A is an inhibitor of CYP17A1, 3β-hydroxysteroid dehydrogenase/Δ⁵⁻⁴ isomerase, and 5α-reductase, and has also been found to act as a competitive antagonist of the AR reportedly comparable to the potent antagonist enzalutamide. However, the initial 5α-reduced metabolite of D4A, 3-keto-5α-abiraterone, is an agonist of the AR, and promotes prostate cancer progression. Its formation can be blocked by the coadministration of dutasteride, a potent and selective 5α-reductase inhibitor.

Abiraterone acetate, via its metabolite abiraterone, has the capacity to lower circulating testosterone levels to less than 1 ng/dL (i.e., undetectable), and these concentrations are much lower than those achieved by castration (20 ng/dL). The addition of abiraterone acetate to castration was found to reduce levels of DHT by 85%, DHEA by 97-98%, and androstenedione by 77-78% relative to castration alone.

Accordingly, provided herein are solid oral dosage forms comprising about 500 mg of abiraterone acetate and having a dissolution profile characterized by one or more of features (a)-(f):

(a) about 43% of said dosage form dissolves after five minutes;

(b) about 71% of said dosage form dissolves after 10 minutes;

(c) about 88% of said dosage form dissolves after 20 minutes;

(d) about 94% of said dosage form dissolves after 30 minutes;

(e) about 98% of said dosage form dissolves after 45 minutes; and,

(f) about 99% of said dosage form dissolves after 60 minutes,

when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

In some embodiments, the dosage forms have a dissolution profile characterized by at least two, at least three, at least four, at least five, or all of (a)-(f) as described above.

Also provided herein are solid oral dosage forms comprising about 500 mg of abiraterone acetate and having a dissolution profile characterized by one or more of features (a)-(f):

(a) about 31% of said dosage form dissolves after five minutes;

(b) about 65% of said dosage form dissolves after 10 minutes;

(c) about 94% of said dosage form dissolves after 20 minutes;

(d) about 98% of said dosage form dissolves after 30 minutes;

(e) about 99% of said dosage form dissolves after 45 minutes; and,

(f) about 99% of said dosage form dissolves after 60 minutes,

when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

In some embodiments, the dosage forms have a dissolution profile characterized by at least two, at least three, at least four, at least five, or all of (a)-(f) as described above.

With respect to all of the embodiments disclosed herein, a dosage form having a dissolution profile in which the dissolution percentages for any given time point are within 80-110% of the stated percentages is considered to be a dosage form of the invention. Thus, for example, a dosage form that exhibits 24.8% dissolution after five minutes would be within the scope of the invention, as would one that exhibits 34.1% dissolution.

Also disclosed are solid oral dosage forms comprising about 500 mg of abiraterone acetate; and a film coating that is positioned on an outer surface of said dosage form, wherein said dosage forms are bioequivalent, when administered orally on an equivalent dose basis) to 250 mg ZYTIGA® abiraterone acetate dosage forms.

Any of the dosage forms of the invention may comprise about 30 to about 50 wt % of said abiraterone acetate. In some embodiments, the dosage forms comprise about 35 to about 45 wt % of said abiraterone acetate. In other embodiments, the dosage forms comprise about 35 wt % of said abiraterone acetate. In other embodiments, the dosage forms comprise about 45 wt % of said abiraterone acetate.

The dosage forms may further comprise one or more of a diluent, a disintegrant, a binder, a surfactant, a glidant, or a lubricant. In some embodiments, the dosage forms further comprise a diluent, a disintegrant, a binder, a surfactant, a glidant, and a lubricant. In some embodiments, the diluent is selected from lactose monohydrate, microcrystalline cellulose, and silicified microcrystalline cellulose. In certain embodiments, the disintegrant is croscarmellose sodium. In certain embodiments, the binder is selected from povidone and hypromellose. In embodiments comprising a surfactant, the surfactant may be sodium lauryl sulfate.

In some embodiments, the instant dosage forms comprise lactose monohydrate, microcrystalline cellulose or silicified microcrystalline cellulose, croscarmellose sodium, povidone or hypromellose, sodium lauryl sulfate, colloidal silicon dioxide, and magnesium stearate.

Certain embodiments may comprise about 22-28 wt % lactose monohydrate, about 16-17 wt % microcrystalline cellulose or about 18-20 wt % silicified microcrystalline cellulose, about 6-7 wt % croscarmellose sodium, about 4-6 wt % povidone or about 1-2 wt % hypromellose, about 4-6 wt % sodium lauryl sulfate, about 0.5-1.5 wt % colloidal silicon dioxide, and about 1-2 wt % magnesium stearate.

In some embodiments, the dosage forms comprise about 35 wt % abiraterone acetate, about 28 wt % lactose monohydrate, about 20 wt % silicified microcrystalline cellulose, about 6 wt % croscarmellose sodium, about 5 wt % povidone, about 1 wt % colloidal silicon dioxide, and about 1.5 wt % magnesium stearate, wherein the combination of each of said components in the dosage form equals 100 wt %.

In some embodiments, the dosage forms comprise about 45 wt % abiraterone acetate, about 23 wt % lactose monohydrate, about 17 wt % microcrystalline cellulose, about 6 wt % croscarmellose sodium, about 2 wt % hypromellose, about 1 wt % colloidal silicon dioxide, and about 1.5 wt % magnesium stearate, wherein the combination of each of said components in the dosage form equals 100 wt %.

With respect to certain embodiments, two of said dosage forms administered orally at substantially the same time exhibit a Cₘₐₓ of about 130 ng/mL.

As used herein, administration of two or more dosage forms at “substantially the same time” refers to the administration to a subject of two or more dosage forms at exactly the same time (i.e., such that the dosage forms are all swallowed simultaneously), or wherein all of the two or more dosage forms are administered within a period of time that would constitute a medically acceptable period of time for administration of a single dosage. For example, the period of time within which all of the two or more dosage forms are ingested may be less than about 30 seconds, one minute, five minutes, seven minutes, 10 minutes, 15 minutes, 20 minutes, 30 minutes, 35 minutes, 40 minutes, 45 minutes, 50 minutes, 55 minutes, 60 minutes, 65 minutes, 70 minutes, 75 minutes, 80 minutes, 90 minutes, 95 minutes, 100 minutes, 105 minutes, 110 minutes, 115 minutes, 2 hours, 2 hours 15 minutes, 2 hours 30 minutes, or 3 hours.

In certain embodiments, two of the present dosage forms administered orally at substantially the same time exhibit a Cₘₐₓ of about 108 ng/mL.

In some embodiments, two of the dosage forms administered orally at substantially the same time exhibit a tₘₐₓ at about 2.0 hours.

In some embodiments, two of the dosage forms administered orally at substantially the same time exhibit an area under the plasma concentration-time curve from time zero to the time of the last quantifiable concentration (AUCₗₐₛₜ) of about 679 ng*h/mL.

In certain embodiments, two of the instant dosage forms administered orally at substantially the same time exhibit an area under the plasma concentration-time curve from time zero to the time of the last quantifiable concentration (AUCₗₐₛₜ) of about 589 ng*h/mL.

In some embodiments, two of the present dosage forms administered orally at substantially the same time exhibit an area under the plasma concentration-time curve extrapolated to infinite time (AUC_{∞}) of about 707 ng*h/mL.

In some embodiments, two of the present dosage forms administered orally at substantially the same time exhibit an area under the plasma concentration-time curve extrapolated to infinite time (AUC_{∞}) of about 600 ng*h/mL.

In certain embodiments, two of the present dosage forms administered orally at substantially the same time exhibit an apparent terminal elimination half-life (t_{1/2}) of about 18.6 hours.

In some embodiments, two of the present dosage forms orally at substantially the same time exhibit an apparent terminal elimination half-life (t_{1/2}) of about 18.1 hours.

The present disclosure also provides methods of reducing pill burden on a subject in need of an abiraterone acetate pharmaceutical regimen comprising orally administering to a subject two dosage forms according to the present disclosure at substantially the same time.

Also provided are methods of treating a subject using an abiraterone acetate pharmaceutical regimen that is bioequivalent to 250 mg ZYTIGA® abiraterone acetate dosage forms when administered orally on an equivalent dose basis, comprising orally administering a dosage form according to the present disclosure.

The present disclosure also provides methods of treating a subject who has prostate cancer comprising orally administering to said subject a dosage form according to the present disclosure.

Also provided are methods of selling a drug product comprising abiraterone acetate, said method comprising selling such drug product, wherein a drug product label for a reference listed drug for such drug product includes instructions for treating non-metastatic castration resistant prostate cancer.

Also disclosed are methods of offering for sale a drug product comprising abiraterone acetate, said method comprising offering for sale such drug product, wherein a drug product label for a reference listed drug for such drug product includes instructions for treating non-metastatic castration resistant prostate cancer.

The present disclosure also provides methods of selling disclosed a drug product comprising abiraterone acetate, said method comprising selling such drug product, wherein the drug product label for a reference listed drug for such drug product comprises metastasis free survival data.

Also disclosed are methods of offering for sale a disclosed drug product comprising abiraterone acetate, said method comprising offering for sale such drug product, wherein the drug product label for a reference listed drug for such drug product comprises metastasis free survival data.

The present disclosure also provides an approved drug product comprising 500 mg abiraterone acetate.

As used herein, the term, “drug product” is product that contains an active pharmaceutical ingredient that has been approved for marketing by a governmental authority, e.g., the U.S. Food and Drug Administration or the similar authority in other countries.

The term “Reference Listed Drug (RLD)” is a drug product to which new generic versions are compared to show that they are bioequivalent. It is also a medicinal product that has been granted marketing authorization by a Member State of the European Union or by the Commission on the basis of a completed dossier, i.e., with the submission of quality, pre-clinical and clinical data in accordance with Articles 8(3), 10a, 10b or 10c of Directive 2001/83/EC and to which the application for marketing authorization for a generic/hybrid medicinal product refers, by demonstration of bioequivalence, usually through the submission of the appropriate bioavailability studies.

In the United States, a company seeking approval to market a generic equivalent must refer to the RLD in its Abbreviated New Drug Application (ANDA). For example, an ANDA applicant relies on the FDA's finding that a previously approved drug product, i.e., the RLD, is safe and effective, and must demonstrate, among other things, that the proposed generic drug product is the same as the RLD in certain ways. Specifically, with limited exceptions, a drug product for which an ANDA is submitted must have, among other things, the same active ingredient(s), conditions of use, route of administration, dosage form, strength, and (with certain permissible differences) labeling as the RLD. The RLD is the listed drug to which the ANDA applicant must show its proposed ANDA drug product is the same with respect to active ingredient(s), dosage form, route of administration, strength, labeling, and conditions of use, among other characteristics. In the electronic Orange Book, there will is a column for RLDs and a column for reference standards. In the printed version of the Orange Book, the RLDs and reference standards are identified by specific symbol.

In Europe, Applicants identify in the application form for its generic/hybrid medicinal product, which is the same as an ANDA or sNDA drug product, the reference medicinal product (product name, strength, pharmaceutical form, MAH, first authorization, Member State/Community), which is synonymous with an RLD, as follows:
1. The medicinal product that is or has been authorized in the EEA, used as the basis for demonstrating that the data protection period defined in the European pharmaceutical legislation has expired. This reference medicinal product, identified for the purpose of calculating expiry of the period of data protection, may be for a different strength, pharmaceutical form, administration route or presentation than the generic/hybrid medicinal product.
2. The medicinal product, the dossier of which is cross-referred to in the generic/hybrid application (product name, strength, pharmaceutical form, MAH, marketing authorization number). This reference medicinal product may have been authorized through separate procedures and under a different name than the reference medicinal product identified for the purpose of calculating expiry of the period of data protection. The product information of this reference medicinal product will, in principle, serve as the basis for the product information claimed for the generic/hybrid medicinal product.
3. The medicinal product (product name, strength, pharmaceutical form, MAH, Member State of source) used for the bioequivalence study(ies) (where applicable).

The different abbreviated approval pathways for drug products under the FD&C Act—the abbreviated approval pathways described in section 505(j) and 505(b)(2) of the FD&C Act (21 U.S.C. 355(j) and 21 U.S.C. 23 355(b)(2), respectively).

According to the FDA (https://www.fda.gov/downloads/Drugs/GuidanceComplianceRegulatoryInformation/Guidances/UCM579751.pdf, the contents of which is incorporated herein by reference), NDAs and ANDAs can be divided into the following four categories:
(1) A “stand-alone NDA” is an application submitted under section 505(b)(1) and approved under section 505(c) of the FD&C Act that contains full reports of investigations of safety and effectiveness that were conducted by or for the applicant or for which the applicant has a right of reference or use.
(2) A 505(b)(2) application is an NDA submitted under section 505(b)(1) and approved under section 505(c) of the FD&C Act that contains full reports of investigations of safety and effectiveness, where at least some of the information required for approval comes from studies not conducted by or for the applicant and for which the applicant has not obtained a right of reference or use.
(3) An ANDA is an application for a duplicate of a previously approved drug product that was submitted and approved under section 505(j) of the FD&C Act. An ANDA relies on FDA's finding that the previously approved drug product, i.e., the reference listed drug (RLD), is safe and effective. An ANDA generally must contain information to show that the proposed generic product (a) is the same as the RLD with respect to the active ingredient(s), conditions of use, route of administration, dosage form, strength, and labeling (with certain permissible differences) and (b) is bioequivalent to the RLD. An ANDA may not be submitted if studies are necessary to establish the safety and effectiveness of the proposed product.
(4) A petitioned ANDA is a type of ANDA for a drug product that differs from the RLD in its dosage form, route of administration, strength, or active ingredient (in a product with more than one active ingredient) and for which FDA has determined, in response to a petition submitted under section 505(j)(2)(C) of the FD&C Act (suitability petition), that studies are not necessary to establish the safety and effectiveness of the proposed drug product.

A scientific premise underlying the Hatch-Waxman Amendments is that a drug product approved in an ANDA under section 505(j) of the FD&C Act is presumed to be therapeutically equivalent to its RLD. Products classified as therapeutically equivalent can be substituted with the full expectation that the substituted product will produce the same clinical effect and safety profile as the prescribed product when administered to patients under the conditions specified in the labeling. In contrast to an ANDA, a 505(b)(2) application allows greater flexibility as to the characteristics of the proposed product. A 505(b)(2) application will not necessarily be rated therapeutically equivalent to the listed drug it references upon approval.

The terms “sale” or “selling” means transferring a drug product, e.g., a pharmaceutical composition or an oral dosage form, from a seller to a buyer.

The term “offering for sale” means the proposal of a sale by a seller to a buyer for a drug product, e.g., a pharmaceutical composition and an oral dosage form.

Routes of Administration and Pharmaceutical Compositions

Therapeutic agents described herein are administered in any suitable manner or suitable formulation. Suitable routes of administration of the therapeutic agents include, but are not limited to, oral and parenteral (e.g., intravenous, subcutaneous, intramuscular). All formulations are in dosages suitable for administration to a human. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The term “safe and effective amount” refers to an amount of an active ingredient that elicits the desired biological or medicinal response in a subject's biological system without the risks outweighing the benefits of such response in accordance with the Federal Food, Drug, and Cosmetic Act, as amended (secs. 201-902,52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “acceptable” with respect to a formulation, composition or ingredient, as used herein, means having no persistent detrimental effect on the general health of the male human being treated.

In some embodiments, administration of a safe and effective amount of the anti-androgen results in no more than a grade 2 adverse event. In other embodiments, administration of a safe and effective amount of anti-androgen results in no more than a grade 3 adverse event. In other embodiments, administration of a safe and effective amount of anti-androgen results in no more than a grade 4 adverse event.

In some embodiments, the anti-androgen is present in a solid dosage form. In some embodiments, the anti-androgen is formulated as a tablet. In some embodiments, the anti-androgen is abiraterone acetate.

The amount of abiraterone acetate that is administered to the subject may be about 500 to about 1500 mg/day, about 600 to about 1300 mg/day, about 700 to about 1200 mg/day, about 800 to about 1200 mg/day, about 900 to about 1100 mg/day, about 950 to about 1050 mg/day, or may be about 500, about 600, about 700, about 750, about 800, about 850, about 875, about 900, about 925, about 950, about 1000, about 1025, about 1050, about 1075, about 1100, or about 1125 mg/day.

In another aspect, described herein are methods of selling an anti-androgen comprising, consisting of, or consisting essentially of placing the anti-androgen into the stream of commerce wherein said anti-androgen includes a package insert that contains instructions for safely and effectively treating prostate cancer using the anti-androgen. In some embodiments, the anti-androgen is a second-generation anti-androgen. In some embodiments, the anti-androgen is abiraterone acetate. In some embodiments, the anti-androgen is enzalutamide In some embodiments, the anti-androgen is apalutamide.

In further aspects, described herein are methods of selling a pharmaceutical composition containing anti-androgen comprising, consisting of, or consisting essentially of placing such pharmaceutical composition into the stream of commerce wherein such pharmaceutical composition includes a package insert that contains instructions for safely and effectively treating prostate cancer using anti-androgen. In some embodiments, the anti-androgen is a second-generation anti-androgen. In some embodiments, the anti-androgen is abiraterone acetate.

In still further aspects, described herein are methods of offering for sale anti-androgen comprising, consisting of, or consisting essentially of offering to place the anti-androgen into the stream of commerce wherein said anti-androgen includes a package insert that contains instructions for safely and effectively treating prostate cancer using the anti-androgen. In some embodiments, the anti-androgen is a second-generation anti-androgen. In some embodiments, the anti-androgen is abiraterone acetate.

### EXAMPLES

The present invention is further defined in the following examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

The examples summarize information on 250-mg and 500-mg film-coated (FC) tablets of abiraterone acetate developed to follow the current commercial uncoated tablet. The new dosage strength (500-mg) was developed to reduce the pill burden. The film coat was introduced to eliminate dust formation and to allow for product differentiation by color. The formulation development is described herein. The pivotal bioequivalence study and the supportive relative bioavailability study are described. These studies compared the oral bioavailability of the 250-mg and 500-mg FC tablets with the commercial 250-mg uncoated tablets of abiraterone acetate.

The examples also summarize the re-validation of the liquid chromatography-tandem mass spectrometry (LC-MS/MS) bioanalytical method for quantification of abiraterone in human plasma used for the measurement of abiraterone in plasma samples obtained in all the biopharmaceutic studies. The bioanalytical method, which uses different blood collection tubes from the previously validated method (i.e., tubes containing sodium ethylenediaminetetracetic acid [Na₂EDTA] and sodium fluoride [NaF] were used instead of tubes containing potassium ethylenediaminetetracetic acid [K₂EDTA] with addition of NaF separately) is described.

Overview. New 250-mg and a 500-mg FC tablets have been developed that are bioequivalent to the commercial 250-mg uncoated tablet (G002) on an equivalent dose basis. The film coat was introduced to eliminate dust formation and to allow for product differentiation from other strengths by color. The new dosage strength of 500-mg was developed to reduce the pill burden.

Initially, three test FC tablet concepts containing a lower sodium lauryl sulfate (SLS) content than the commercial uncoated tablet were developed and tested in an exploratory relative bioavailability study: a 250-mg FC tablets with 0.5% SLS (G012), a 500-mg FC tablets with 0.5% SLS (G011), and a 500-mg FC tablets with 2% SLS (G013). The commercial uncoated tablet contains 4% SLS. The results showed that the systemic exposure to abiraterone was lower for all 3 FC tablets relative to the commercial uncoated tablet. This exploratory study provided useful information regarding the effect of SLS on the bioavailability of abiraterone, leading to the successful development of final FC tablet concepts evaluated in subsequent studies.

As a result of the information obtained in the exploratory study, two different 250-mg FC tablets were developed and tested in another relative bioavailability study: a 250-mg FC tablet (G004) made through high-shear granulation and a 250-mg FC tablet (G022) made through fluid-bed granulation. Beige film coat was applied to both tablets (G004 and G022).

Two different 500-mg FC tablet concepts were developed and tested in the relative bioavailability study: a 500-mg FC tablet (G005) made through high-shear granulation and a 500-mg FC tablet (G023) made through fluid-bed granulation. Purple film coat was applied to both tablets (G005 and G023). The composition of the different clinical formulations is presented in Table 1A, below.

TABLE 1A

| | | G002 | G004* | G022 | G005 | G023* |
|---|---|---|---|---|---|---|
| Formulation Strength | | 250-mg | 250-mg | 250-mg | 500-mg | 500-mg |
| Ingredient | Function | % w/w | % w/w | % w/w | % w/w | % w/w |
| Abiraterone Acetate | Active | 34.97 | 34.97 | 44.64 | 34.97 | 44.64 |
| Lactose Monohydrate | Diluent | 27.78 | 27.78 | 22.61 | 27.78 | 22.61 |
| Microcrystalline | Diluent | 19.75 | 19.75 | NA | 19.75 | NA |
| Cellulose | | | | | | |
| Silicified | | | | | | |
| Microcrystalline | Diluent | NA | NA | 17.00 | NA | 16.50 |
| Cellulose | | | | | | |
| Croscarmellose | Disintegrant | 6.00 | 6.00 | 7.00 | 6.00 | 7.00 |
| Sodium | | | | | | |
| Povidone (K29/K32) | Binder | 5.00 | 5.00 | NA | 5.00 | NA |
| Hypromellose 2910, | Binder | NA | NA | 1.50 | NA | 1.50 |
| 15 mPas | | | | | | |
| SLS | Surfactant | 4.00 | 4.00 | 5.00 | 4.00 | 5.50 |
| | Granulating | | | | | |
| Purified Water | Fluid | NA | NA | NA | NA | NA |
| Colloidal Silicon | Glidant | 1.00 | 1.00 | 0.75 | 1.00 | 0.75 |
| Dioxide | | | | | | |
| Magnesium Stearate | Lubricant | 1.50 | 1.50 | 1.50 | 1.50 | 1.50 |
| Total | | 100 | 100 | 100 | 100 | 100 |

NA = not applicable; SLS = sodium lauryl sulfate; w/w = weight/weight.
*New formulations

Table 1B provides the formulation composition for the 250 mg tablets, expressed in terms of the quantity (mg) of each component per unit.

TABLE 1B
Quantity per Unit (mg)

| Component | Function | G004 | G002 |
|---|---|---|---|
| Abiraterone Acetate | Active | 250.00 | 250.00 |
| Lactose Monohydrate (80M) | Diluent | 198.65 | 198.65 |
| MCC (Avicel PH101) | Diluent | 107.25 | 107.25 |
| Croscarmellose Sodium | Disintegrant | 21.45 | 21.45 |
| (Acdisol) | Binder | 35.75 | 35.75 |
| Povidone (Plasdone K29/32) | | | |
| Sodium Lauryl sulfate | Surfactant | 28.60 | 28.60 |
| Purified water^{a} | Granulation Fluid | N/A | N/A |
| MCC (Avicel PH101) | Diluent | 33.97 | 33.97 |
| Croscarmellose Sodium | Disintegrant | 21.45 | 21.45 |
| (Acdisol) | | | |
| Collodial Silicon Dioxide | Glidant | 7.15 | 7.15 |
| (Cab-O-Sil M5P) | Lubricant | 10.73 | 10.73 |
| Magnesiumstearate | | | |
| Nominal Weight: | | 715.00 | 715.00 |
| Opadry II85F170011 Beige | Film-Coating | 21.45 | — |
| Powder | | | |
| Nominal Weight: | | 736.45 | 715.00 |

^{a}Not present in final product

Table 1C provides the formulation composition for an inventive 500 mg tablet (G023), expressed in terms of the quantity (mg) and weight percentage of each component per unit.

TABLE 1C

| | | | |
|---|---|---|---|
| | | Quantity per | % |
| | | Unit | Compo- |
| Component | Function | (mg) | sition |

Internal phase

| | | | |
|---|---|---|---|
| Abiraterone Acetate | Active | 500.0 | 44.64 |
| Lactose Monohydrate 200 mesh | Filler | 253.2 | 22.61 |
| Croscarmellose Sodium | Disintegrant | 22.4 | 2.00 |

| | |
|---|---|
| Granules | 775.6 |

Binder solution

| | | | |
|---|---|---|---|
| Hypromellose 2910 15 mPa · s | Binder | 16.8 | 1.50 |
| Sodium Lauryl Sulphate | Surfactant | 5.6 | 0.50 |
| Purified water^{a} | Solvent | 560.0 | NA |
| Binder solution | | 22.4 | |

External phase

| | | | |
|---|---|---|---|
| Silicified Microcrystalline | Diluent | 184.8 | 16.50 |
| Cellulose (HD90) | | | |
| Croscarmellose Sodium | Disintegrant | 56.0 | 5.00 |
| Sodium Lauryl Sulphate | Surfactant | 56.0 | 5.00 |
| Colloidal Anhydrous Silica | Glidant | 8.4 | 0.75 |
| Magnesium Stearate | Lubricant | 16.8 | 1.50 |
| External phase | | 322 | |
| Nominal Weight: | | 1120.0 | |

Coating

| | | | |
|---|---|---|---|
| Opadry II85F90093 Purple | Film-Coating | 33.6 | 3.00 |
| Powder | | | |
| Purified water^{a} | Solvent | 134.4 | NA |
| Nominal Weight: | | 1153.6 | |

^{a}Not present in final product

An overview of all formulation batches is provided in Table 1D, below.

TABLE 1D
Formulations Used Across Biopharmaceutic Studies

| Formulation | | | Drug Product | |
|---|---|---|---|---|
| Number | Strength | Description | Batch Number | Clinical Study |
| G002 | 250-mg | Uncoated tablet; Commercial formulation | FFTN | 212082PCR1006 |
| G011 | 500-mg | FC tablet; New composition | 12C08 | 212082PCR1006 |
| G012 | 250-mg | FC tablet; New composition | 12C06 | 212082PCR1006 |
| G013 | 500-mg | FC tablet; New composition | 12C13 | 212082PCR1006 |
| G002 | 250-mg | Uncoated tablet; Commercial formulation | 4367707 | 212082PCR1010 |
| G022 | 250-mg | FC tablet; New composition | 4367700 | 212082PCR1010 |
| G023 | 500-mg | FC tablet; New composition | 4367701 | 212082PCR1010 |
| G004 | 250-mg | FC tablet, Same composition as | 4367702 | 212082PCR1010 |
| | | commercial formulation | | |
| G005 | 500-mg | FC tablet; Same composition as | 4367703 | 212082PCR1010 |
| | | commercial formulation | | |
| G002 | 250-mg | Uncoated tablet; Commercial formulation | PFTF | 212082PCR1007 |
| G004 | 250-mg | FC tablet; Same composition as | SBCN | 212082PCR1007 |
| | | commercial formulation | | |
| G023 | 500-mg | FC tablet; New composition | 4207 | 212082PCR1007 |

FC = film-coated

The SLS content (%w/w) in formulations G004 and G005 is the same as the commercial uncoated tablet. Formulations G022 and G023 have higher SLS content (%w/w) than the commercial uncoated tablet.

Based on the results of the concept study, the 250-mg FC tablet (G004) and 500-mg FC tablet (G023) were selected for use in the pivotal bioequivalence study (212082PCR1007), in which bioequivalence with the commercial 250-mg uncoated tablet (G002) was demonstrated for both tablets. Details of study 212082PCR1007 are provided infra.

The 250-mg FC tablet (G004) and 500-mg FC tablet (G023) were associated with beneficial results. Apart from the film-coat, the 250-mg FC tablet (G004) is identical to the 250-mg uncoated tablet (G002) in terms of composition and manufacturing process (high-shear granulation).

The 500-mg FC tablet (G023) is manufactured using fluid bed granulation. The excipient composition was adjusted to meet the manufacturability requirements of the new manufacturing process, to reduce the tablet weight, and to meet the desired quality profile for dissolution and bioavailability.

The new 250-mg FC tablets (G004) have the same composition and are produced at the same manufacturing site, using the same manufacturing process and equipment of the same operating principle and design as the pivotal clinical bioequivalence batch of formulation G004.

The manufacturing of the 500-mg FC tablet (G023) can be performed at small and at large commercial scale. The pivotal clinical batch used in the bioequivalence study and the primary stability batches were produced at small commercial scale. The dissolution profiles of the primary stability batches and a batch produced at large commercial scale have been shown to be comparable to the dissolution profile of the pivotal bioequivalence batch by means of f2 comparison (f2>50). Therefore, it was concluded that drug product batches manufactured at small commercial scale and at large commercial scale are of comparable quality and perform similar to the bioequivalence batch.

### Example 1

### In Vitro Release

Dissolution methods similar to the approved dissolution method for the commercially available 250-mg uncoated tablets (G002) were used for the 250-mg (G004) and the 500-mg (G023) FC tablets. The parameters of the dissolution methods are summarized in Table 2, below:

TABLE 2

| Parameter | Conditions | |
|---|---|---|
| Tablet Strength | 250-mg | 500-mg |
| Dissolution Apparatus: | Basket (USP type 1, Ph. Eur., JP) | Paddle (USP type 2, Ph. Eur., JP) |
| Dissolution Medium | 37.0 ± 0.5° C. | 37.0 ± 0.5° C. |
| Temperature: | | |
| Dissolution Medium Volume: | 900 mL | 900 mL |
| Dissolution Medium: | 56.5 mM phosphate buffer pH 4.5 with | 56.5 mM phosphate buffer pH 4.5 with |
| | 0.25% (w/v) SLS | 0.25% (w/v) SLS |
| Rotation Speed: | 75 rpm | 75 rpm |
| Sample Filter: | Syringe filter 10 μm pore size, | Syringe filter 0.45-μm pore size, |
| | regenerated cellulose membrane | regenerated cellulose membrane |
| Assay Method: | UHPLC with UV detection at 254 nm | UHPLC with UV detection at 254 nm |

USP = United States Pharmacopeia; JP = Japan; Ph. Eur. = European Pharmacopoeia; SLS = sodium lauryl sulfate; UHPLC = ultra high-performance liquid chromatography; UV = ultraviolet; w/v = weight/volume.

The following parameters of the approved dissolution method for the 250-mg uncoated tablet (G002) were modified for the 250-mg FC tablets (G004):
Dissolution apparatus: from paddle to basket;
Rotation speed: from 50 to 75 rpm to avoid coning;
Assay method: high performance liquid chromatography (HPLC) was replaced by UHPLC, which is more efficient than the current platform system used.

During the course of development, the dissolution method parameters for the 500-mg FC tablets (G023) were changed compared to the dissolution parameters used for the commercial 250-mg uncoated tablets (G002). The following parameters have been modified:
Rotation speed: from 50 to 75 rpm to avoid coning;
Filter: smaller pore size of filter (0.45 μm) because particles of coating material do not dissolve in the dissolution medium and need to be removed by filtration to prevent blocking of the column of the ultra high performance liquid chromatography (UHPLC) system;
Assay method: HPLC was replaced by UHPLC, which is more efficient than the current platform system used.

In vitro dissolution data for the 250-mg (G004) and 500-mg (G023) FC tablet batches used in clinical studies are provided below in Table 3.

TABLE 3
In Vitro Dissolution Data for To-be-marketed 500-mg FC Tablet Used in Biopharmaceutic Studies

| | Batch/ | | | Collection times, Mean % Dissolved | | | | | | |
|---|---|---|---|---|---|---|---|---|---|---|
| | Formulation | Dissolution | Media | 5 | 10 | 20 | 30 | 45 | 60 | |
| Dosage Form | Number | Apparatus | Temperature | min | min | min | min | min | min | Study ID |
| 250-mg FC tablet | 4367702/G004 | Basket (USP | 56.5 mM phosphate buffer | 22 | 50 | 87 | 98 | 100 | 100 | 212082PCR1010 |
| | | type 1, Ph. Eur., | pH 45 with 0.25% (w/v) | | | | | | | |
| | | JP), 10 mesh | SLS 37.0 ± 0.5° C. | | | | | | | |
| | | baskets | | | | | | | | |
| 250-mg FC tablet | SBCN/G004 | Basket (USP | 56.5 mM phosphate buffer | 30 | 59 | 94 | 97 | 98 | 98 | 212082PCR1007 |
| | | type 1, Ph. Eur., | pH 45 with 0.25% (w/v) | | | | | | | |
| | | JP), 10 mesh | SLS 37.0 ± 0.5° C. | | | | | | | |
| | | baskets | | | | | | | | |
| 500-mg FC tablet | 4367701(13D23)/ | Paddle (USP | 56.5 mM phosphate buffer | 43 | 71 | 88 | 94 | 98 | 99 | 212082PCR1010 |
| | G023 | type 2, Ph. Eur., | pH 45 with 0.25% (w/v) | | | | | | | |
| | | JP) | SLS 37.0 ± 0.5° C. | | | | | | | |
| 500-mg FC tablet | 4027/G023 | Paddle (USP | 56.5 mM phosphate buffer | 31 | 65 | 94 | 98 | 99 | 99 | 212082PCR1007 |
| | | type 2, Ph. Eur., | pH 45 with 0.25% (w/v) | | | | | | | |
| | | JP) | SLS 37.0 ± 0.5° C. | | | | | | | |

FC = film-coated; USP = United States Pharmacopeia; Ph. Eur. = European Pharmacopoeia; JP = Japan; SLS = sodium lauryl sulfate; w/v = weight/volume.

### Example 2

### Biopharmaceutical Studies

The pivotal bioequivalence study 212082PCR1007 and the supportive relative bioavailability study 212082PCR1010 are summarized in Table 4, below.

TABLE 4
Phase 1 Biopharmaceutical Studies Summarized in Module 2.7.1

| | | | |
|---|---|---|---|
| | | | Dosage Regimen and |
| Study Number | Number of | | Formulation Numbers (Drug |
| (Section of Module 2.7.1) | Subjects | Study Description | Product Batch Numbers) |

Relative Bioavailability

| | | | |
|---|---|---|---|
| 212082PCR1010 | 32 | A single-dose, open-label, | Abiraterone acetate 1,000 mg |
| (Section [00116]) | | randomized, 8-sequence, | Single oral dose |
| | | 4-period, 5-treatment, crossover | G002 (4367707): 4 × 250-mg |
| | | design in healthy subjects | G022 (4367700): 4 × 250-mg |
| | | | G023 (4367701): 2 × 500-mg |
| | | | G004 (4367702): 4 × 250-mg |
| | | | G005 (4367703): 2 × 500-mg |

Bioequivalence

| | | | |
|---|---|---|---|
| 212082PCR1007 | 102 | A single-dose, open-label, | Abiraterone acetate 1,000 mg |
| (Section [00121]) | | randomized, 6-sequence, | Single oral dose |
| | | 3-period, 3-treatment, crossover | G002 (PFTF): 4 × 250-mg |
| | | design in healthy subjects | G004 (SBCN): 4 × 250-mg |
| | | | G023 (4207): 2 × 500-mg |

An overview of these biopharmaceutical studies is presented in Table 5 and a tabular listing of bioavailability pharmacokinetic parameters across the two studies is presented in Table 6. More details are given infra.

TABLE 5

| | | | | | Formulation/Drug Product Batch | |
|---|---|---|---|---|---|---|
| Study ID | Objective(s) | Subjects | Dose | Route | Number | Pivotal |
| 212082PCR1010 | Relative | Healthy | 1,000 mg | Oral | 4 × 250-mg commercial uncoated | |
| | bioavailability | | Single dose | | tablets (G002)/4367707 | |
| | (FC tablets vs. | | | | 4 × 250-mg FC tablets (G022)/ | |
| | commercial | | | | 4367700 | |
| | coated tablets) | | | | 2 × 500-mg FC tablets (G023)/ | |
| | | | | | 4367701 | |
| | | | | | 4 × 250-mg FC tablets (G004)/ | |
| | | | | | 4367702 | |
| | | | | | 2 × 500-mg FC tablets(G005)/ | |
| | | | | | 4367703 | |
| 212082PCR1007 | Bioequivalence | Healthy | 1,000 mg | Oral | 4 × 250-mg commercial uncoated | X |
| | (FC tablets vs. | | Single dose | | tablets (G002)/PFTF | |
| | commercial | | | | 4 × 250-mg FC tablets (G004)/ | |
| | coated tablets) | | | | SBCN | |
| | | | | | 2 × 500-mg FC tablets (G023)/4207 | |

FC = film-coated

TABLE 6

| Tablet Strength mg | | | Median (Range) | | Arithmetic Mean (SD) | |
|---|---|---|---|---|---|---|
| (Total Dose mg) of | | | tₘₐₓ | | Cₘₐₓ | |
| Abiraterone Acetate | Formulation | N | (h) | | (ng/mL) | |
| 250 (1,000) | Reference (Commercial uncoated | 32 | 2.00 | (1.00-8.00) | 111 | (64.3) |
| | tablets, G002) | | | | | |
| 250 (1,000) | Test (FC tablets, G022) | 23 | 3.00 | (1.50-4.02) | 121 | (96.3) |
| 500 (1,000) | Test (FC tablets, G023) | 23 | 2.00 | (1.00-4.00) | 130 | (80.8) |
| 250 (1,000) | Test (FC tablets, G004) | 22 | 1.51 | (1.00-4.00) | 131 | (159) |
| 500 (1,000) | Test (FC tablets, G005) | 22 | 2.00 | (1.08-6.00) | 108 | (87.5) |
| 250 (1,000) | Reference (Commercial uncoated | 100 | 2.00 | (1.00-6.02) | 107 | (76.2) |
| | tablets, G002) | | | | | |
| 250 (1,000) | Test (FC tablets, G004) | 100 | 2.00 | (1.00-6.03) | 99.4 | (63.6) |
| 500 (1,000) | Test (FC tablets, G023) | 101 | 2.00 | (1.00-12.00) | 112 | (73.6) |

| Tablet Strength mg | Arithmetic Mean (SD) | | | | | | |
|---|---|---|---|---|---|---|---|
| (Total Dose mg) of | AUCₗₐₛₜ | | AVC_{∞} | | t_{1/2} | | |
| Abiraterone Acetate | (ng*h/mL) | | (ng*h/mL) | | (h) | | Study ID |
| 250 (1,000) | 615 | (343) | 626 | (347) | 18.6 | (5.53) | 212082PCR1010 |
| 250 (1,000) | 671 | (509) | 6 [symbol] 3 | (516) | 18.0 | (4.83) | 212082PCR1010 |
| 500 (1,000) | 679 | (366) | 707 | (379)^{a} | 18.6 | (6.26)^{b} | 212082PCR1010 |
| 250 (1,000) | 643 | (610) | 654 | (615) | 19.5 | (5.16) | 212082PCR1010 |
| 500 (1,000) | 589 | (414) | 600 | (419) | 18.1 | (6.38) | 212082PCR1010 |
| 250 (1,000) | 629 | (4 [symbol] 8) | 641 | (472) [symbol] | 16.3 | (4.3)^{b} | 212082PCR1007 |
| 250 (1,000) | 553 | (323) | 566 | (342) [symbol] | 16.6 | (8.0)^{b} | 212082PCR1007 |
| 500 (1,000) | 632 | (415) | 641 | (417) | 16.5 | (4.3) | 212082PCR1007 |

AUC_{∞} = area under the plasma concentration-time curve extrapolated to infinite time; AUCₗₐₛₜ = area under the plasma concentration-time curve from time zero to the time of the last quantifiable concentration; Cₘₐₓ = maximum observed plasma concentration; t_{1/2} apparent terminal elimination half-life; tₘₐₓ = time to reach the maximum observed plasma concentration, FC = Film-coated; SD = standard deviation.
^{a}N = 22
^{b}N = 99 [symbol]
indicates data missing or illegible when filed

Relative Bioavailability and Bioequivalence. Study 212082PCR1010 was conducted to evaluate the relative bioavailability of four abiraterone acetate FC tablets (250-mg: G004 and G005; 500-mg: G022 and G023) compared with the commercial uncoated tablets (250-mg, G002) at a single dose of 1,000 mg.

The results of this study led to the selection of the 250-mg FC tablet (G004) and 500-mg FC tablet (G023) for the 212082PCR1007 study to assess their bioequivalence with respect to the commercial uncoated tablet.

### Example 3

### Bioanalytical Methods

Measurement of Abiraterone Concentrations in Plasma. Abiraterone concentrations in plasma were measured using an LC-MS/MS method, consisting of liquid/liquid extraction with methyl-t-butyl ether, chromatography on an UPLC and detection via multiple reaction monitoring. This method was developed at Pharmaceutical Research Associates (PRA, Assen, The Netherlands) in 2010.

In 2012, the bioanalytical method was re-validated at PRA to accommodate use of blood sampling tubes containing Na₂EDTA and NaF. Previously, human blood samples were collected in K₂EDTA sampling tubes with addition of 0.5 mol/L NaF separately to plasma in a 1:10 NaF to plasma ratio. Incurred sample reproducibility and a partial validation to include a different injection volume were also performed. The bioanalytical method for abiraterone in plasma was validated according to globally accepted procedures discussed in international meetings (including the United States [US] Food and Drug Administration Guidance for Industry: Bioanalytical Method Validation, 2001). These procedures were reflected in the Standard Operating Procedures in PRA. All acceptance criteria as specified in these procedures were met. The validated plasma abiraterone concentrations range were 0.200 to 500 ng/mL, which were the same as previously validated in 2010.

A tabulated summary of specific details of the bioanalytical method can be found in Table 7, below:

TABLE 7
Overview of Validated Bioanalytical Methods

| | | Matrix, | | | | |
|---|---|---|---|---|---|---|
| Lab | Year | Anticoagulant | Extraction | | Mass Spectrometry | |
| Identification | Validated | Stabilizer | Procedure | Chromatography | Conditions | Remarks |
| PRA | 2010 | Plasma, | Liquid-liquid | Waters Acquity UPLC | MRM mode | Used in previous |
| International, | | K₂EDTA + | Extraction | BEH C18 (1.7 μm, 2.1 × | abiraterone acetate: | submission |
| Assen, | | NaF | methyl-t- | 50 mm) gradient 0.1% | m/z 392.3 → 332.0 | |
| Netherlands | | | butyl ether | Formic acid in water, | abiraterone: | |
| | | | | methanol/acetonitrile 1/1 | m/z 350.2→ 156.0 | |
| PRA | 2012 | Plasma, | Liquid-liquid | Waters Acquity UPLC | MRM mode | Partial validation |
| International, | | Na₂EDTA + | Extraction | BEH C18 (1.7 μm, 2.1 × 50 | abiraterone acetate: | added to include |
| Assen, | | NaF | methyl-t- | mm) gradient 0.1% Formic | m/z 392.3 → 332.0 | different injection |
| Netherlands | | | butyl ether | acid in water, | abiraterone: | volume |
| | | | | methanol/acetonitrile 1/1 | m/z 350.2→ 156.0 | |

BEH = Ethylene Bridged Hybrid; Na₂EDTA = sodium ethylenediaminetetracetic acid; K₂EDTA = potassium ethylenediaminetetracetic acid; MRM = multiple reaction monitoring; NaF = sodium fluoride; PRA = Pharmaceutical Research Associates; UPLC = ultra performance liquid chromatography.

### Example 4

### Summary of Results of Individual Studies

The relative bioavailability and bioequivalence studies summarized in this Module were single-dose, open-label, randomized, cross-over study designs conducted in healthy adult male subjects between 18 and 55 years of age, inclusive. The Body Mass Index for all subjects was within 18.5 to 30 kg/m², inclusive. A crossover design was used to permit intra-subject comparison and eliminate potential confounding factors due to inter-subject variability. A washout period of at least 7 days was required between subsequent treatment periods.

Abiraterone acetate tablets were administered orally with 240 mL of noncarbonated water, after an overnight fast of at least 10 hours. No food was allowed to be ingested for at least 4 hours post-dose for both studies. Subjects were advised to remain seated, standing or ambulatory for at least 1 hour following dose administration. Serial blood samples for pharmacokinetic analysis were collected from pre-dose up to 96 hours post-dose to determine abiraterone concentrations.

In previous studies, plasma concentrations of abiraterone acetate were below the lower limit of quantitation. Because of this, assessment of abiraterone acetate from plasma samples was not feasible; therefore, blood samples were collected to measure plasma concentrations of abiraterone only. Plasma pharmacokinetic parameters of abiraterone were calculated based on actual sampling times, relative to abiraterone acetate dosing, using conventional non-compartmental methods. Subjects that had sufficient data for calculation of at least one pharmacokinetic parameter were included in the pharmacokinetic analysis.

Statistical analyses were performed on log-transformed pharmacokinetic parameters from subjects who completed all treatment periods, using statistical models as specified in each study protocol. For each pharmacokinetic parameter, the mean difference in log-transformed data between the test group and the reference group and the associated 90% confidence intervals (CI) were calculated. The ratios of geometric mean, expressed as a percent, and the associated 90% CI were generated by back-transformation on the original scale. Bioequivalence between treatments was declared if the 90% CI of geometric mean ratios for area under the plasma concentration-time curve (AUC) and maximum observed plasma concentration (Cₘₐₓ) were within established bioequivalence limits of 80.00% to 125.00%.

Figures and tables presented in this section were generated specifically for this document based upon the exact data in the study reports. Numeric values presented in the tables in this section represent arithmetic mean±standard deviation (SD) for the individual treatments; tₘₐₓ values are presented as median (range).

Relative Bioavailability Study of Four New Film-coated Tablets of Abiraterone Acetate (Study 212082PCR1010). Summary: Both 250-mg FC tablets demonstrated similar systemic exposure to abiraterone compared with the uncoated tablet. Formulation G004 employed the current manufacturing process of the uncoated commercial tablet. For the two 500-mg-FC tablets, G023 showed more comparable systemic exposure than G005 with respect to the uncoated tablet.

Study Design and Objectives: Study 212082PCR1010 was a single-dose, single-center, randomized, open-label, 4-period, 8-sequence, 5-treatment, crossover study in healthy male subjects. The primary objective of the study was to evaluate the relative bioavailability of abiraterone following administration of four test abiraterone acetate FC tablets with respect to the current commercial uncoated tablets. Thirty-two subjects were randomized to 1 of 8 treatment sequences (AEBD, BACE, CBDA, EDAC, DECA, EADB, ABEC, BCAD), where:
Treatment A (reference)—4×250-mg abiraterone acetate current commercial uncoated tablets (G002), administered orally as a single dose under fasted conditions
Treatment B (test)—4×250-mg abiraterone acetate FC tablets (G022), administered orally as a single dose under fasted conditions
Treatment C (test)—2×500-mg abiraterone acetate FC tablets (G023), administered orally as a single dose under fasted conditions
Treatment D (test)—4×250 mg abiraterone acetate FC tablets (G004), administered orally as a single dose under fasted conditions
Treatment E (test)—2×500 mg abiraterone acetate FC tablets (G005), administered orally as a single dose under fasted conditions

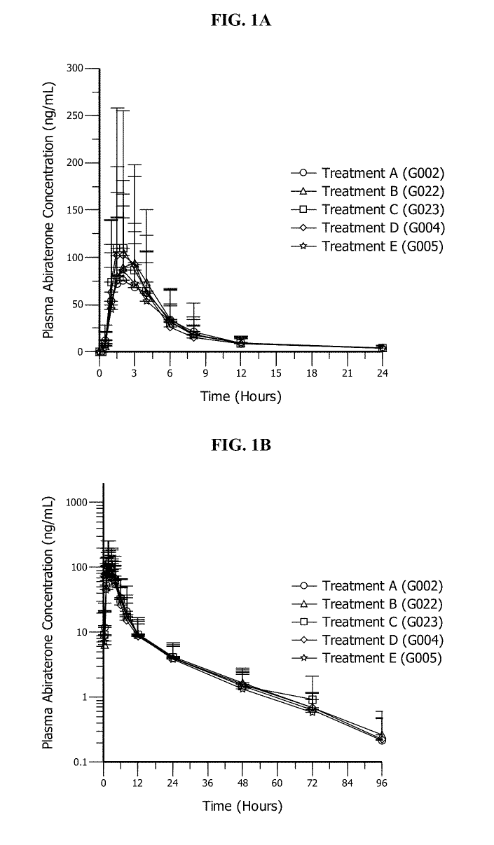

Results: The mean plasma concentration-time profiles of abiraterone following single-dose administration of abiraterone acetate tablets are presented in FIGS. 1A and 1B. Mean plasma pharmacokinetic parameters of abiraterone and statistical comparisons of these parameters are presented in Table 8, below.

TABLE 8
Arithmetic Mean (SD) Plasma Pharmacokinetic Parameters of Abiraterone and Geometric
Mean Ratios and Their Associated 90% Confidence Intervals (Study 212082PCR1010)

| | Treatment A | | Treatment B | | Treatment C | | Treatment D | | Treatment E | |
|---|---|---|---|---|---|---|---|---|---|---|
| | Uncoated | | Film-coated | | Film-coated | | Film-coated | | Film-coated | |
| | 4 × 250-mg | | 4 × 250-mg | | 2 × 500-mg | | 4 × 250-mg | | 2 × 500-mg | |
| | (G002) | | (G022) | | (G023) | | (G004) | | (G005) | |
| PK Parameter, Unit | N = 32 | | N = 23 | | N = 23 | | N = 22 | | N = 22 | |
| Cₘₐₓ, ng/mL | 111 | (64.3) | 121 | (96.3) | 130 | (80.8) | 131 | (159) | 108 | (87.5) |
| tₘₐₓ, h^{a} | 2.00 | (1.00-8.00) | 3.00 | (1.50-4.02) | 2.00 | (1.00-4.00) | 1.51 | (1.00-4.00) | 2.00 | (1.08-6.00) |
| AUCₗₐₛₜ, ng*h/mL | 615 | (343) | 671 | (509) | 679 | (366) | 643 | (610) | 589 | (414) |
| AUC_{∞}, ng*h/mL | 626 | (347) | 683 | (516) | 707 | (379)^{b} | 654 | (615) | 600 | (419) |
| t_{1/2}, h | 18.6 | (5.53) | 18.0 | (4.83) | 18.6 | (6.26)^{b} | 19.5 | (5.16) | 18.1 | (6.38) |

| | Ratio (%) of Geometric Means (90% CI) | | | | | | | |
|---|---|---|---|---|---|---|---|---|
| | Test/Reference (%) | | | | | | | |
| | Treatment B/A | | Treatment C/A | | Treatment D/A | | Treatment E/A | |
| PK Parameter | N = 21 | | N = 22 | | N = 22 | | N = 22 | |
| Cₘₐₓ | 101.23 | (81.62-125.55) | 103.67 | (83.98-127.98) | 95.75 | (77.62-118.13) | 90.66 | (73.36-112.03) |
| AUCₗₐₛₜ | 99.07 | (82.90-118.41) | 103.38 | (86.83-123.08) | 96.09 | (80.75-114.34) | 91.68 | (76.93-109.25) |
| AUC_{∞} | 99.96 | (83.45-119.73)^{c} | 101.94 | (85.30-121.84)^{d} | 94.52 | (79.20-112.79)^{d} | 91.23 | (76.58-108.68) |

^{a}Median (range)
^{b}N = 22
^{c}N = 20
^{d}N = 21
AUC_{∞} = area under the plasma concentration-time curve extrapolated to infinite time; AUCₗₐₛₜ = area under the plasma concentration-time curve from time zero to the time of the last quantifiable concentration; Cₘₐₓ = maximum observed plasma concentration; t_{1/2} = mean apparent terminal half-life; tₘₐₓ = time to reach the maximum observed plasma concentration; CI = confidence interval; SD = standard deviation.

The median tₘₐₓ and mean apparent terminal half-life (t_{1/2}) of plasma abiraterone were similar between the four test FC tablets and the uncoated tablet. In comparison to the uncoated tablet, both 250-mg FC tablets demonstrated comparable systemic exposure (Cₘₐₓ, AUCₗₐₛₜ, and AUC_{∞}) to abiraterone. Formulation G004 was the to-be-marketed tablet formulation of choice because the same manufacturing process used to make the current commercial uncoated tablet, except for the film-coating, could be used to make the new FC formulation. In case of the 500-mg FC tablets, G005 showed lower relative bioavailability to abiraterone than G023. Therefore, G023 was chosen since it would have higher probability of demonstrating bioequivalence.

Inter-subject variability (coefficient of variation [CV]) for Cₘₐₓ, AUCₗₐₛₜ, and AUC_{∞} ranged from 55.4% to 57.9% for Treatment A, from 75.5% to 79.6% for Treatment B, from 53.6% to 62.2% for Treatment C, from 94.9% to 121% for Treatment D, and from 69.8% to 81.0% for Treatment E. Intra-subject CV for Cₘₐₓ, AUCₗₐₛₜ, and AUC_{∞} were 45.8%, 37.3%, and 37.1%, respectively.

Bioequivalence Study of the to-be-marketed 250-mg and 500-mg Film-coated Tablets versus the Current Commercial Uncoated Tablets of Abiraterone Acetate (Study 212082PCR1007). Summary: The new to-be-marketed 250-mg FC tablets (G004) and 500-mg FC tablets (G023) were bioequivalent with the current commercial 250-mg uncoated tablets of abiraterone acetate (G002).

Study Design and Objectives: Study 212082PCR1007 was a single-dose, single-center, randomized, open-label, 3-period, 6-sequence, 3-treatment, crossover study in healthy male subjects. The primary objective was to assess the bioequivalence of the to-be-marketed 250-mg FC tablet and 500-mg FC tablet with respect to the current commercial uncoated tablet at a single dose of 1,000 mg. One hundred and two (102) subjects were randomized to 1 of 6 treatment sequences (ABC, BCA, CAB, ACB, BAC, CBA), where:
Treatment A (reference)—4×250-mg abiraterone acetate current commercial uncoated tablets (G002), administered orally as a single dose under fasted conditions
Treatment B (test)—4×250 mg abiraterone acetate FC tablets (G004), administered orally as a single dose under fasted conditions
Treatment C (test)—2×500 mg abiraterone acetate FC tablets (G023), administered orally as a single dose under fasted conditions

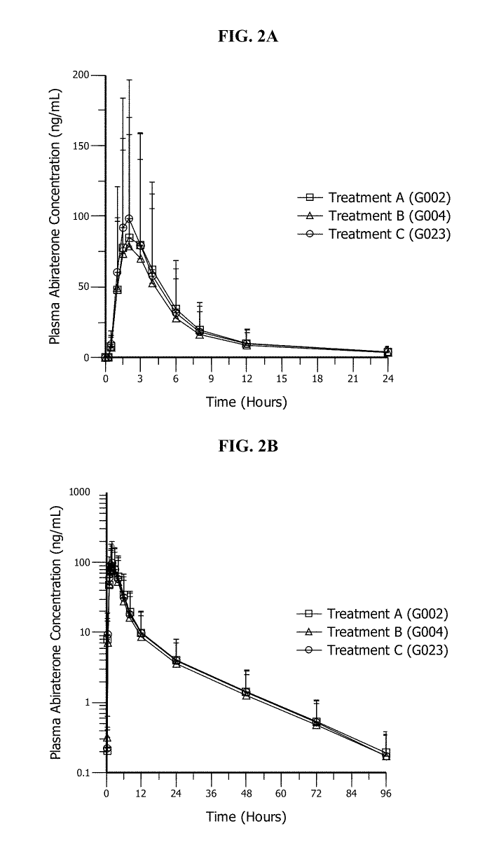

Results: The mean plasma concentration-time profiles of abiraterone following single-dose administration of abiraterone acetate tablets are presented in FIGS. 2A and 2B. Mean plasma pharmacokinetic parameters of abiraterone and their statistical comparisons are present in Table 9, below.

TABLE 9
Arithmetic Mean (SD) Plasma Pharmacokinetic Parameters
of Abiraterone and Geometric Mean Ratios and Their Associated
90% Confidence Intervals (Study 212082PCR1007)

| | Treatment A | | Treatment B | | Treatment C | |
|---|---|---|---|---|---|---|
| | Uncoated | | Film-coated | | Film-coated | |
| | 4 × 250-mg | | 4 × 250-mg | | 2 × 500-mg | |
| | (G002) | | (G004) | | (G023) | |
| PK Parameter, unit | N = 100 | | N = 100 | | N = 101 | |
| Cₘₐₓ, ng/mL | 107 | (76.2) | 99.4 | (63.6) | 112 | (73.6) |
| tₘₐₓ, h^{a} | 2.00 | (1.00-6.02) | 2.00 | (1.00-6.03) | 2.00 | (1.00-12.00) |
| AUCₗₐₛₜ, ng*h/mL | 629 | (468) | 553 | (323) | 632 | (415) |
| AUC_{∞}, ng*h/mL | 641 | (472)^{b} | 566 | (342)^{b} | 641 | (417) |
| t_{1/2}, h | 16.3 | (4.3)^{b} | 16.6 | (8.0)^{b} | 16.5 | (4.3) |

| | Ratio (%) of Geometric Means (90% CI) | | | |
|---|---|---|---|---|
| | Test/Reference (%) | | | |
| | Treatment B/A | | Treatment C/A | |
| | N = 99 | | N = 99 | |
| Cₘₐₓ | 92.96 | (84.98-101.69) | 102.77 | (93.95-112.43) |
| AUCₗₐₛₜ | 90.68 | (84.42-97.40) | 100.77 | (93.82-108.24) |
| AUC_{∞} | 90.86 | (84.63-97.55)^{c} | 100.46 | (93.57-107.86)^{c} |

^{a}Median (range)
^{b}N = 99
^{c}N = 98
AUC_{∞} = area under the plasma concentration-time curve extrapolated to infinite time; AUCₗₐₛₜ = area under tire plasma concentration-time curve from time zero to the time of the last quantifiable concentration; Cₘₐₓ = maximum observed plasma concentration; t_{1/2} = apparent terminal elimination half-life; tₘₐₓ = time to reach the maximum observed plasma concentration; CI = confidence interval; SD = standard deviation.

The median tₘₐₓ was identical for all treatment groups (2 hours). The mean t_{1/2} of abiraterone was similar for all treatment groups (16.3 to 16.6 hours). The 90% CI of the geometric mean ratios between the test and reference tablets were contained within the 80.00% to 125.00% range for Cₘₐₓ, AUCₗₐₛₜ, and AUC_{∞}. Therefore, it demonstrates that the 250-mg and 500-mg FC tablets are bioequivalent with the current commercial uncoated tablets.

Inter-subject variability (CV) for Cₘₐₓ, AUCₗₐₛₜ, and AUC_{∞} ranged from 71.2% to 74.4% for the 4×250-mg current commercial uncoated tablets (Treatment A), from 58.4% to 64.0% for the 4×250-mg FC tablets (Treatment B), and from 65.1% to 65.7% for the 2×500-mg FC tablets (Treatment C). Intra-subject CV for Cₘₐₓ, AUCₗₐₛₜ, and AUC_{∞} were 39.6%, 31.2%, and 30.8%, respectively.

### Example 5

### Comparison and Analyses of Results Across Studies

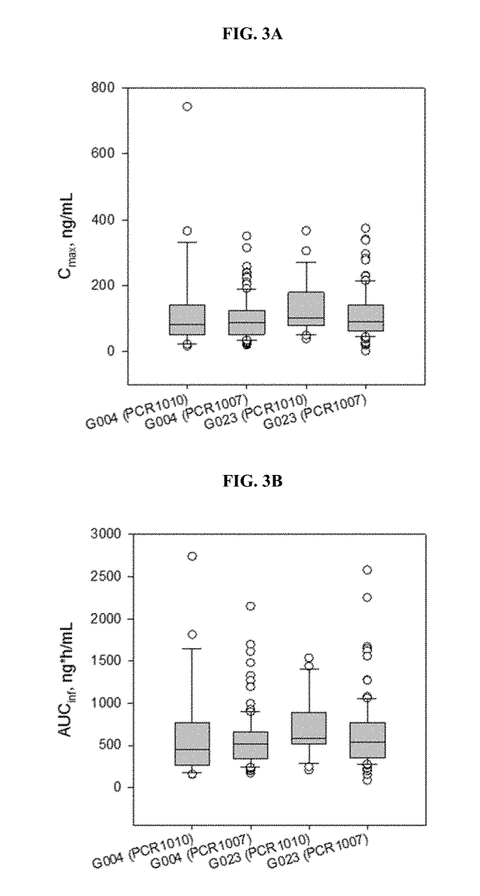

Plasma Abiraterone Pharmacokinetics Following Single-Dose Administration of Abiraterone Acetate under Fasted Conditions. The inventive 250-mg (G004) and 500-mg (G023) FC tablet formulations were evaluated in 212082PCR1010 and 212082PCR1007 studies in healthy male subjects under fasted conditions. Box plots of key pharmacokinetic parameters (Cₘₐₓ and AUC_{∞}) from these two studies are presented in FIG. 3. Mean Cₘₐₓ and AUC_{∞} values were comparable across the two studies for each formulation, showing reproducibility. Pharmacokinetic parameters were also similar between the two formulations evaluated.

Impact of Formulation Changes: Film-coated Tablets Versus the Current Commercial Uncoated Tablets. The bioavailability study (212082PCR1010) demonstrated comparable bioavailability between the current commercial uncoated tablet and inventive FC tablet formulations. Two formulations (G004 and G005) have the same SLS content as the commercial uncoated tablet. The other two formulations (G022 and G023) have slightly higher SLS content compared to the commercial uncoated tablet. Based on the results of study 212082PCR1010, a 250-mg FC tablet (G004) with the same composition and manufacturing process as the commercial uncoated tablet and a 500-mg FC tablet (G023) with a new composition were selected to be evaluated in the pivotal bioequivalence study 212082PCR1007. This pivotal bioequivalence study confirmed that the proposed to-be-marketed 250-mg and 500-mg FC tablets were bioequivalent to the current commercial 250-mg uncoated tablet of abiraterone acetate. The change in excipient composition for the 500-mg FC tablet (G023) and film-coat have no impact on the bioavailability of abiraterone acetate.

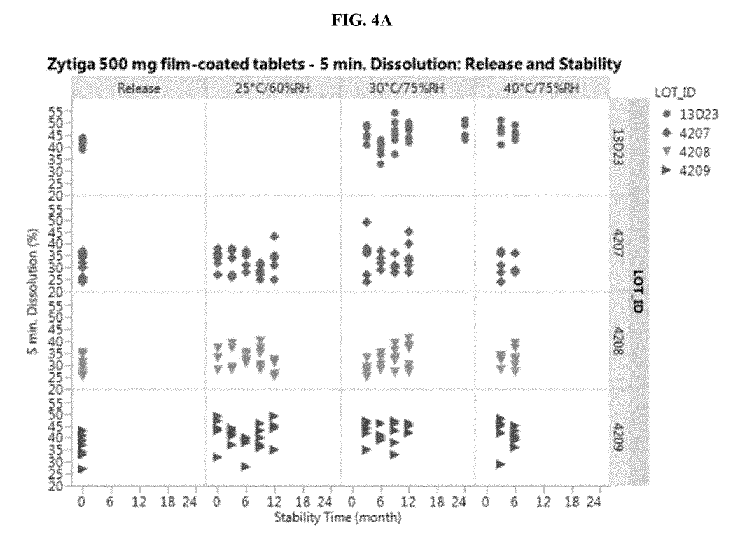

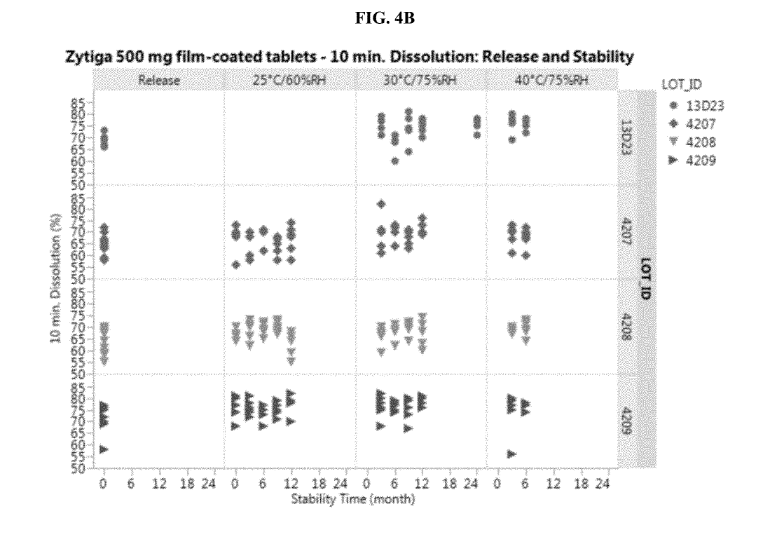

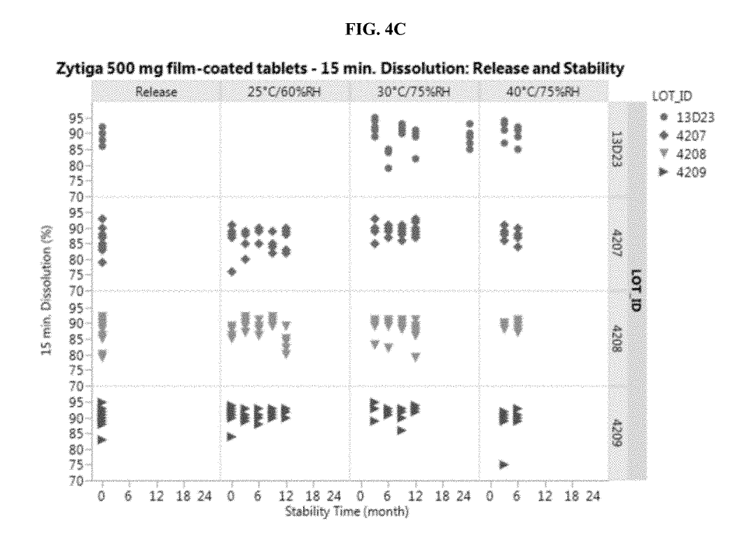

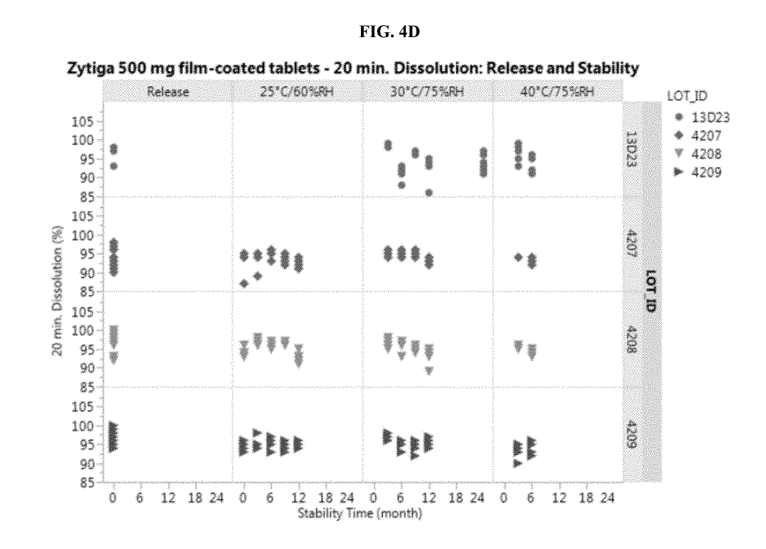

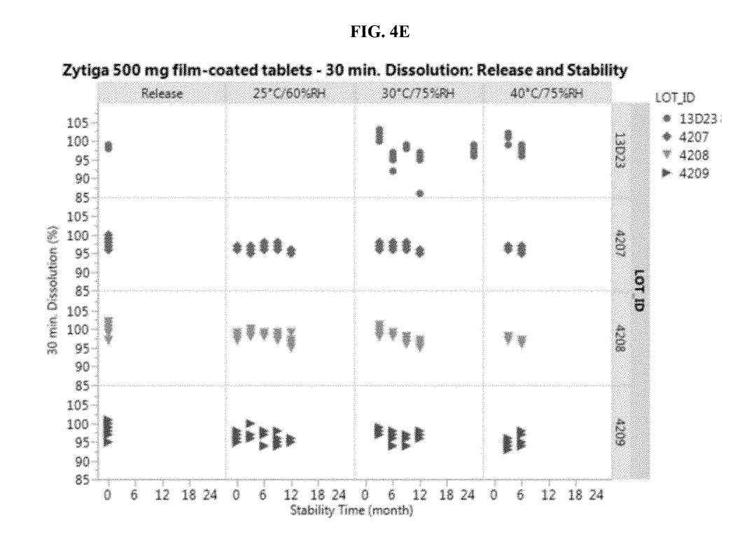

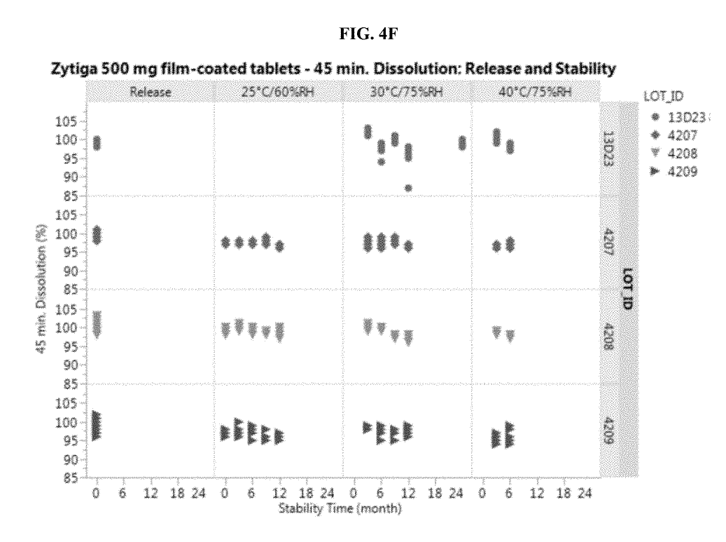

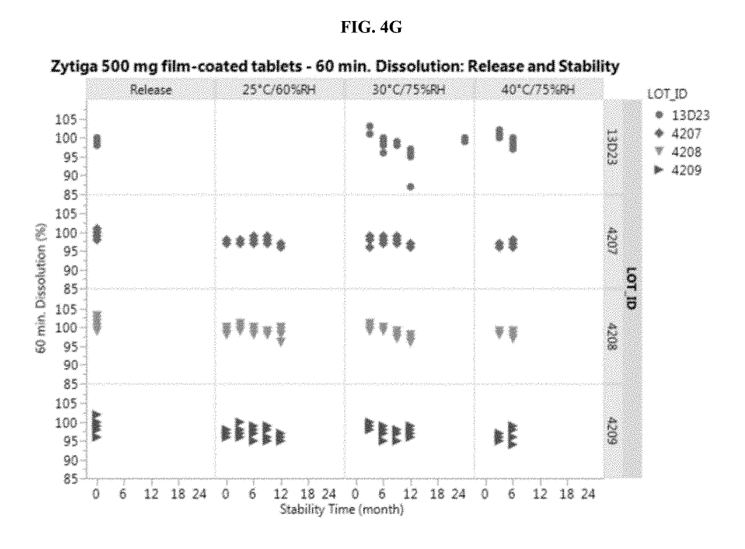

Additional Information. FIGS. 4A-4G illustrate the results of an assessment of release and stability by the inventive 500 mg film-coated tablet dosage forms at specified time points (5, 10, 15, 20, 30, 45, and 60 minutes, respectively).

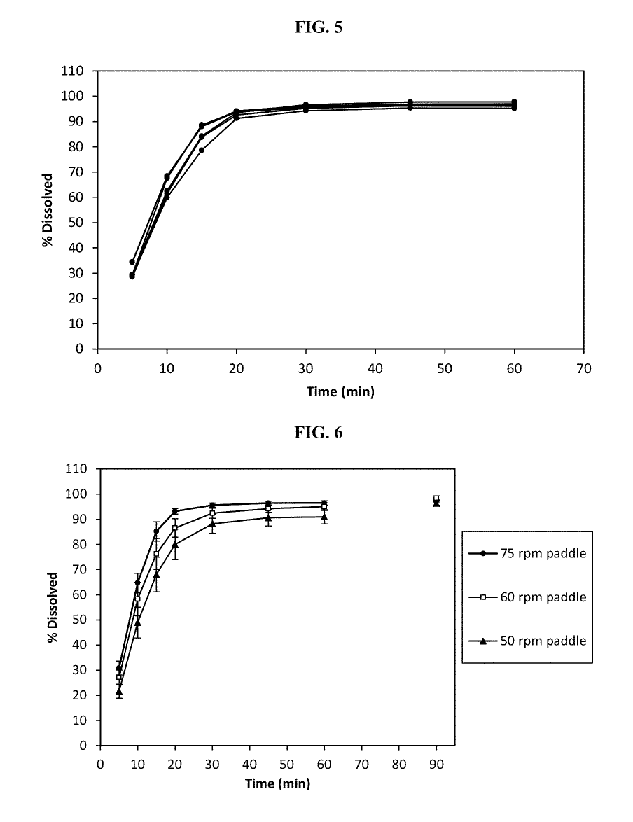

FIG. 5 illustrates a typical dissolution profile of the 500-mg FC tablets (G023) obtained using the proposed dissolution method. In particular, the figure shows individual dissolution profiles of batch 4207 measured with the chosen dissolution method. Table 10 likewise provides the obtained dissolution profiles.

TABLE 10

| Time | | | | | | | | | |
|---|---|---|---|---|---|---|---|---|---|
| (min) | Vessel 1 | Vessel 2 | Vessel 3 | Vessel 4 | Vessel 5 | Vessel 6 | Avg | SD | RSD (%) |
| 5 | 28 | 29 | 29 | 34 | 29 | 34 | 31 | 2.74 | 8.87 |
| 10 | 61 | 63 | 68 | 68 | 60 | 68 | 65 | 3.80 | 5.87 |
| 15 | 84 | 84 | 89 | 88 | 79 | 88 | 85 | 3.85 | 4.52 |
| 20 | 93 | 94 | 94 | 94 | 91 | 94 | 93 | 1.12 | 1.20 |
| 30 | 95 | 97 | 95 | 96 | 94 | 96 | 96 | 0.83 | 0.87 |
| 45 | 96 | 98 | 96 | 97 | 95 | 96 | 96 | 0.77 | 0.80 |
| 60 | 96 | 98 | 96 | 97 | 95 | 97 | 97 | 0.86 | 0.89 |

Avg = average;
SD = standard deviation;
RSD = relative standard deviation

FIG. 6 illustrates average dissolution profiles and SD of batch 4207 of the 500-mg FC tablets (G023) as measured with a paddle apparatus at different rotation speeds (50, 60, and 75 rpm) using 900 mL phosphate buffer of pH 4.5 containing 0.25% of SLS at 37° C. (N=6). Tables 11-14 likewise provide the obtained dissolution profiles.

TABLE 11
Average Dissolution Results of Batch 4207 Measured with a Paddle
Apparatus at Different Rotation Speeds (50, 60, and 75 rpm) Using
900 mL Phosphate Buffer of pH 4.5 Containing
0.25% of SLS at 37° C. (N = 6)

| | Amount Dissolved (%) | | | | | | | | |
|---|---|---|---|---|---|---|---|---|---|
| | 50 rpm | | | 60 rpm | | | 75 rpm | | |
| Time | | | RSD | | | RSD | | | RSD |
| (min) | Avg | SD | (%) | Avg | SD | (%) | Avg | SD | (%) |
| 5 | 22 | 2.73 | 12.61 | 27 | 3.00 | 11.07 | 31 | 2.74 | 8.87 |
| 10 | 49 | 6.09 | 12.45 | 58 | 6.67 | 11.44 | 65 | 3.80 | 5.87 |
| 15 | 68 | 6.86 | 10.08 | 76 | 6.14 | 8.05 | 85 | 3.85 | 4.52 |
| 20 | 80 | 6.07 | 7.59 | 87 | 3.66 | 4.22 | 93 | 1.12 | 1.20 |
| 30 | 88 | 3.87 | 4.39 | 92 | 1.99 | 2.15 | 96 | 0.83 | 0.87 |
| 45 | 91 | 3.27 | 3.61 | 94 | 1.47 | 1.56 | 96 | 0.77 | 0.80 |
| 60 | 91 | 2.81 | 3.09 | 95 | 1.48 | 1.56 | 97 | 0.86 | 0.89 |
| 90^{a} | 96 | 0.81 | 0.84 | 98 | 0.89 | 0.91 | 97 | 0.57 | 0.59 |

^{a}An infinity sample was taken at this time point after 30 minutes agitation at 250 rpm
Avg = average;
SD = standard deviation;
RSD = relative standard deviation

TABLE 12
Individual Dissolution Results of Batch 4207 Measured with a
Paddle Apparatus at 50 rpm Using 900 mL Phosphate Buffer
of pH 4.5 Containing 0.25% of SLS at 37° C.

| | Amount Dissolved (%) | | | | | |
|---|---|---|---|---|---|---|
| Time | Vessel | | Vessel | | | |
| (min) | 1 | Vessel 2 | 3 | Vessel 4 | Vessel 5 | Vessel 6 |
| 5 | 23 | 18 | 25 | 19 | 24 | 21 |
| 10 | 50 | 43 | 55 | 44 | 57 | 45 |
| 15 | 71 | 61 | 77 | 64 | 74 | 62 |
| 20 | 81 | 72 | 89 | 78 | 84 | 75 |
| 30 | 86 | 85 | 93 | 90 | 92 | 84 |
| 45 | 89 | 89 | 94 | 91 | 95 | 86 |
| 60 | 89 | 90 | 94 | 91 | 95 | 87 |
| 90^{a} | 95 | 97 | 97 | 97 | 97 | 95 |

^{a}An infinity sample was taken at this time point after 30 minutes agitation at 250 rpm

TABLE 13
Individual Dissolution Results of Batch 4207 Measured with a
Paddle Apparatus at 60 rpm Using 900 mL Phosphate Buffer
of pH 4.5 Containing 0.25% of SLS at 37° C.

| | Amount Dissolved (%) | | | | | |
|---|---|---|---|---|---|---|
| Time | Vessel | | Vessel | | | |
| (min) | 1 | Vessel 2 | 3 | Vessel 4 | Vessel 5 | Vessel 6 |
| 5 | 28 | 28 | 26 | 32 | 23 | 26 |
| 10 | 66 | 55 | 55 | 67 | 52 | 54 |
| 15 | 80 | 71 | 75 | 87 | 72 | 72 |
| 20 | 88 | 82 | 86 | 92 | 88 | 84 |
| 30 | 93 | 89 | 93 | 94 | 94 | 92 |
| 45 | 95 | 91 | 95 | 95 | 95 | 94 |
| 60 | 96 | 92 | 95 | 95 | 96 | 96 |
| 90^{a} | 99 | 97 | 99 | 99 | 98 | 99 |

^{a}An infinity sample was taken at this time point after 30 minutes agitation at 250 rpm

TABLE 14
Individual Dissolution Results of Batch 4207 Measured with a
Paddle Apparatus at 75 rpm Using 900 mL Phosphate Buffer
of pH 4.5 Containing 0.25% of SLS at 37° C.

| | Amount Dissolved (%) | | | | | |
|---|---|---|---|---|---|---|
| Time | Vessel | | Vessel | | | |
| (min) | 1 | Vessel 2 | 3 | Vessel 4 | Vessel 5 | Vessel 6 |
| 5 | 28 | 29 | 29 | 34 | 29 | 34 |
| 10 | 61 | 63 | 68 | 68 | 60 | 68 |
| 15 | 84 | 84 | 89 | 88 | 79 | 88 |
| 20 | 93 | 94 | 94 | 94 | 91 | 94 |
| 30 | 95 | 97 | 95 | 96 | 94 | 96 |
| 45 | 96 | 98 | 96 | 97 | 95 | 96 |
| 60 | 96 | 98 | 96 | 97 | 95 | 97 |
| 90^{a} | 97 | 98 | 97 | 97 | 97 | 97 |

^{a}An infinity sample was taken at this time point after 30 minutes agitation at 250 rpm

### Example 6

### Drug Product Stability Changes

To evaluate if the selected dissolution method is sufficiently discriminating towards potential changes in dissolution behavior during product storage, tablets were stored under severe stress conditions of temperature and humidity. The tablets were stored in open conditions (unpacked) for 14 days at different temperatures and humidity conditions (50° C./10% RH, 70° C./10% RH, 70° C./40% RH, and 70° C./75% RH).

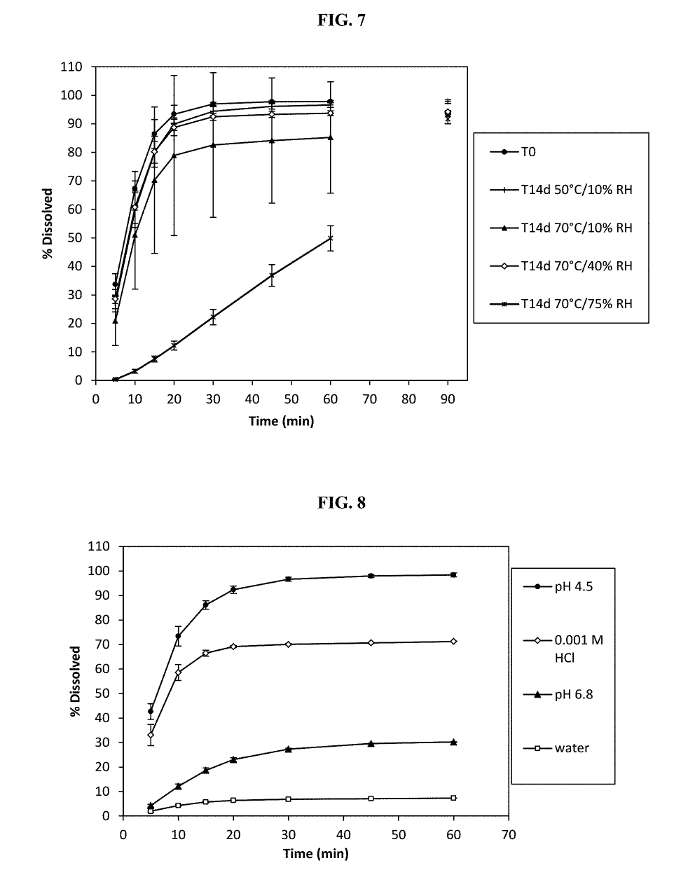

The average dissolution results are shown in FIG. 7 and Table 15.

TABLE 15

| | Amount Dissolved (%) | | | | | | | | | | | | | | |
|---|---|---|---|---|---|---|---|---|---|---|---|---|---|---|---|
| | | | | T14d | | | T14d | | | T14d | | | T14d | | |
| | T0 | | | 50° C./10% RH | | | 70° C./10% RH | | | 70° C./70% RH | | | 70° C./75% RH | | |
| Time | | | RSD | | | RSD | | | RSD^{a} | | | RSD | | | RSD |
| (min) | Avg | SD | (%) | Avg | SD | (%) | Avg | SD^{a} | (%) | Avg | SD | (%) | Avg | SD | (%) |
| 5 | 34 | 3.77 | 11.21 | 27 | 2.92 | 10.80 | 21 | 8.60 | 41.24 | 29 | 3.37 | 11.81 | 0 | 0.13 | 38.32 |
| 10 | 67 | 6.03 | 8.97 | 60 | 6.15 | 10.29 | 51 | 18.99 | 37.24 | 61 | 5.65 | 9.29 | 3 | 0.66 | 20.27 |
| 15 | 86 | 5.07 | 5.87 | 80 | 3.82 | 4.77 | 70 | 25.68 | 36.56 | 80 | 5.41 | 6.75 | 8 | 1.02 | 13.59 |
| 20 | 93 | 3.13 | 3.36 | 90 | 2.18 | 2.43 | 79 | 28.06 | 35.57 | 89 | 2.92 | 3.29 | 12 | 1.57 | 12.82 |
| 30 | 97 | 0.60 | 0.62 | 94 | 1.58 | 1.68 | 83 | 25.36 | 30.70 | 92 | 1.27 | 1.37 | 22 | 2.71 | 12.23 |
| 45 | 98 | 0.33 | 0.34 | 96 | 0.90 | 0.93 | 84 | 21.98 | 26.12 | 93 | 1.09 | 1.16 | 37 | 3.79 | 10.29 |
| 60 | 98 | 0.38 | 0.39 | 97 | 0.86 | 0.89 | 85 | 19.51 | 22.89 | 94 | 0.86 | 0.92 | 50 | 4.41 | 8.86 |
| 90^{b} | — | — | — | 98 | 0.63 | 0.64 | 93 | 0.63 | 0.67 | 94 | 0.46 | 0.49 | 92 | 1.64 | 1.79 |

^{a}Very high SD and RSD caused by an outlier due to the film-coating that impeded the tablet from disintegrating
^{b}An infinity sample was taken at this time point after 30 minutes agitation at 250 rpm
Avg = average;
SD = standard deviation;
RSD = relative standard deviation
— = Not tested

Testing Different Dissolution Media. In order to find the most suitable dissolution medium for this drug product, several media of different pH containing a standard amount of Sodium Lauryl Sulfate (SLS) were tested. The medium for which the highest solubility is obtained was selected in order to limit the needed amount of surfactant. Subsequently, the concentration of the surfactant was further optimized for the sink conditions and discriminating capabilities of the dissolution method.

The dissolution profiles, which are presented in FIG. 8 and Table 16, were determined for the proposed 500-mg FC tablet (G023) using the selected dissolution parameters (paddle, 900 mL at 37.0±0.5° C.) with a rotation speed of 75 rpm in different aqueous media. It can be seen that the highest solubility of the drug substance is obtained in the phosphate buffer pH 4.5. Therefore, this pH is selected for additional experiments to optimize the concentration of surfactant.

TABLE 16
Average Dissolution Results of Batch JNJ-212082-n006-00399 Measured with a Paddle
Apparatus at 75 rpm Using 900 mL Dissolution Media with a Different pH Containing
0.25% SLS at 37° C. (N = 6)

| | Amount Dissolved (%) | | | | | | | | | | | |
|---|---|---|---|---|---|---|---|---|---|---|---|---|
| | Phosphate buffer | | | | | | Phosphate | | | | | |
| | pH 4.5 | | | 0.001M HCl | | | buffer pH 6.8 | | | Water | | |
| | 0.25% SLS | | | 0.25% SLS | | | 0.25% SLS | | | 0.25% SLS | | |
| | | | RSD | | | RSD | | | RSD | | | RSD |
| (min) | Avg | SD | (%) | Avg | SD | (%) | Avg | SD | (%) | Avg | SD | (%) |
| 5 | 43 | 3.22 | 7.56 | 33 | 4.30 | 13.01 | 4 | 0.57 | 13.36 | 2 | 0.28 | 14.62 |
| 10 | 73 | 4.05 | 5.52 | 59 | 3.25 | 5.55 | 12 | 0.95 | 7.79 | 4 | 0.35 | 8.14 |
| 15 | 86 | 1.75 | 2.03 | 66 | 1.19 | 1.78 | 19 | 0.99 | 5.30 | 6 | 0.26 | 4.61 |
| 20 | 92 | 1.48 | 1.60 | 69 | 0.44 | 0.64 | 23 | 0.73 | 3.19 | 6 | 0.16 | 2.48 |
| 30 | 97 | 0.85 | 0.88 | 70 | 0.28 | 0.40 | 27 | 0.37 | 1.36 | 7 | 0.11 | 1.57 |
| 45 | 98 | 0.62 | 0.63 | 71 | 0.24 | 0.35 | 30 | 0.16 | 0.53 | 7 | 0.11 | 1.51 |
| 60 | 98 | 0.78 | 0.79 | 71 | 0.20 | 0.28 | 30 | 0.12 | 0.39 | 7 | 0.09 | 1.22 |

Avg = average;
SD = standard deviation;
RSD = relative standard deviation

A pH value of 4.5 was selected to be used for the dissolution medium which is the same as that used for the 250-mg uncoated tablet (G002).

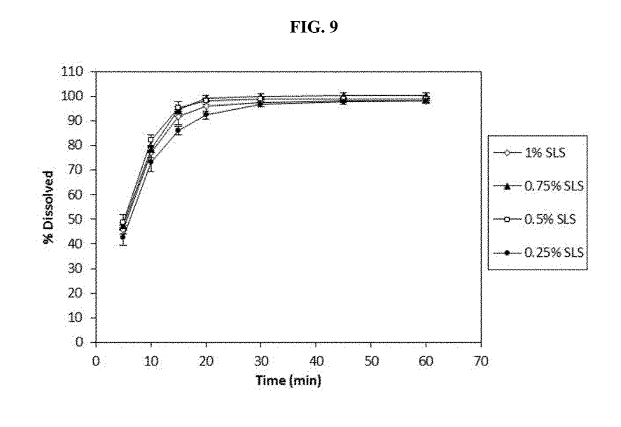

Dissolution profiles were also determined in the selected media containing different % SLS. The dissolution profiles are presented in FIG. 9 and the average dissolution results are presented in Table 17. The profiles were obtained using the selected dissolution parameters (paddle, 900 mL medium at 37.0±0.5° C.) with a rotation speed of 75 rpm.

TABLE 17
Average Dissolution Results of Batch JNJ-212082-n006-00399 Measured with a Paddle
Apparatus at 75 rpm Using 900 mL Phosphate Buffer pH 4.5 Containing Different %
SLS at 37° C.

| | Amount Dissolved (%) | | | | | | | | | | | |
|---|---|---|---|---|---|---|---|---|---|---|---|---|
| | 0.25% SLS | | | 0.5% SLS | | | 0.75% SLS | | | 1% SLS | | |
| | (N = 6) | | | (N = 3) | | | (N = 3) | | | (N = 3) | | |
| Time | | | RSD | | | RSD | | | RSD | | | RSD |
| (min) | Avg | SD | (%) | Avg | SD | (%) | Avg | SD | (%) | Avg | SD | (%) |
| 5 | 43 | 3.22 | 7.56 | 49 | 3.06 | 6.27 | 48 | 1.05 | 2.21 | 46 | 1.78 | 3.90 |
| 10 | 73 | 4.05 | 5.52 | 82 | 2.11 | 2.56 | 79 | 0.81 | 1.03 | 77 | 2.38 | 3.08 |
| 15 | 86 | 1.75 | 2.03 | 95 | 2.53 | 2.66 | 95 | 0.74 | 0.79 | 92 | 3.06 | 3.34 |
| 20 | 92 | 1.48 | 1.60 | 98 | 1.34 | 1.36 | 99 | 1.11 | 1.12 | 96 | 2.77 | 2.88 |
| 30 | 97 | 0.85 | 0.88 | 99 | 0.95 | 0.96 | 100 | 1.13 | 1.13 | 97 | 1.70 | 1.74 |
| 45 | 98 | 0.62 | 0.63 | 99 | 0.72 | 0.73 | 100 | 1.06 | 1.05 | 98 | 1.15 | 1.17 |
| 60 | 98 | 0.78 | 0.79 | 99 | 0.86 | 0.87 | 100 | 1.11 | 1.11 | 98 | 1.03 | 1.05 |

Avg = average;
SD = standard deviation;
RSD = relative standard deviation

Dissolution profiles were also determined using the selected dissolution parameters (900 mL phosphate buffer of pH 4.5 with 0.25% SLS at 37° C. in a paddle apparatus) with different rotations speeds, and results are presented in FIG. 10 and Table 18.

TABLE 18
Average Dissolution Results of Batch 4207 Measured with a Paddle
Apparatus at Different Rotation Speeds (50, 60, and 75 rpm)
Using 900 mL Phosphate Buffer of pH 4.5 Containing 0.25%
of SLS at 37° C. (N = 6)

| | Amount Dissolved (%) | | | | | | | | |
|---|---|---|---|---|---|---|---|---|---|
| | 50 rpm | | | 60 rpm | | | 75 rpm | | |
| Time | | | RSD | | | RSD | | | RSD |
| (min) | Avg | SD | (%) | Avg | SD | (%) | Avg | SD | (%) |
| 5 | 22 | 2.73 | 12.61 | 27 | 3.00 | 11.07 | 31 | 2.74 | 8.87 |
| 10 | 49 | 6.09 | 12.45 | 58 | 6.67 | 11.44 | 65 | 3.80 | 5.87 |
| 15 | 68 | 6.86 | 10.08 | 76 | 6.14 | 8.05 | 85 | 3.85 | 4.52 |
| 20 | 80 | 6.07 | 7.59 | 87 | 3.66 | 4.22 | 93 | 1.12 | 1.20 |
| 30 | 88 | 3.87 | 4.39 | 92 | 1.99 | 2.15 | 96 | 0.83 | 0.87 |
| 45 | 91 | 3.27 | 3.61 | 94 | 1.47 | 1.56 | 96 | 0.77 | 0.80 |
| 60 | 91 | 2.81 | 3.09 | 95 | 1.48 | 1.56 | 97 | 0.86 | 0.89 |
| 90^{a} | 96 | 0.81 | 0.84 | 98 | 0.89 | 0.91 | 97 | 0.57 | 0.59 |

^{a}An infinity sample was taken at this time point after 30 minutes agitation at 250 rpm
Avg = average;
SD = standard deviation;
RSD = relative standard deviation

Table 19, below, provides further individual and average dissolution results of G023 Batch 4207 measured with the proposed G023 method (Paddle 75 rpm).

TABLE 19

| Time | Amount Dissolved (%) | | | | | | | | | | | | | | |
|---|---|---|---|---|---|---|---|---|---|---|---|---|---|---|---|
| (min) | V1 | V2 | V3 | V4 | V5 | V6 | V7 | V8 | V9 | V10 | V11 | V12 | Avg | SD | RSD (%) |
| 5 | 36 | 34 | 30 | 32 | 26 | 26 | 35 | 36 | 37 | 25 | 24 | 30 | 31 | 4.76 | 15.35 |
| 10 | 66 | 67 | 64 | 65 | 59 | 58 | 72 | 70 | 72 | 65 | 63 | 63 | 65 | 4.48 | 6.89 |
| 15 | 84 | 84 | 85 | 84 | 83 | 79 | 93 | 90 | 90 | 88 | 87 | 83 | 86 | 3.88 | 4.51 |
| 20 | 90 | 91 | 92 | 91 | 94 | 93 | 98 | 97 | 97 | 96 | 96 | 93 | 94 | 2.73 | 2.90 |
| 30 | 97 | 96 | 98 | 97 | 98 | 97 | 100 | 99 | 99 | 98 | 98 | 98 | 98 | 1.08 | 1.11 |
| 45 | 98 | 98 | 99 | 98 | 99 | 98 | 101 | 100 | 99 | 99 | 99 | 99 | 99 | 0.90 | 0.91 |
| 60 | 98 | 98 | 99 | 98 | 99 | 99 | 101 | 100 | 100 | 99 | 99 | 100 | 99 | 0.94 | 0.95 |

Avg = average;
SD = standard deviation;
RSD = relative standard deviation

Table 20, below, provides individual and average dissolution results of G023 Batch 4208 measured with the proposed G023 method (Paddle 75 rpm).

TABLE 20

| Time | Amount Dissolved (%) | | | | | | | | | | | | | | |
|---|---|---|---|---|---|---|---|---|---|---|---|---|---|---|---|
| (min) | V1 | V2 | V3 | V4 | V5 | V6 | V7 | V8 | V9 | V10 | V11 | V12 | Avg | SD | RSD (%) |
| 5 | 31 | 29 | 27 | 35 | 29 | 29 | 35 | 25 | 25 | 26 | 34 | 26 | 29 | 3.74 | 12.91 |
| 10 | 69 | 70 | 61 | 69 | 64 | 67 | 68 | 59 | 55 | 58 | 68 | 59 | 64 | 5.25 | 8.20 |
| 15 | 91 | 92 | 86 | 90 | 85 | 89 | 90 | 85 | 79 | 86 | 88 | 80 | 87 | 4.11 | 4.73 |
| 20 | 99 | 100 | 96 | 99 | 96 | 99 | 98 | 96 | 93 | 97 | 96 | 92 | 97 | 2.45 | 2.53 |
| 30 | 102 | 102 | 99 | 101 | 100 | 101 | 100 | 100 | 97 | 101 | 101 | 99 | 100 | 1.42 | 1.42 |
| 45 | 102 | 103 | 99 | 101 | 100 | 102 | 101 | 101 | 98 | 101 | 102 | 100 | 101 | 1.40 | 1.39 |
| 60 | 102 | 103 | 100 | 101 | 100 | 102 | 101 | 100 | 99 | 101 | 102 | 100 | 101 | 1.16 | 1.15 |

Avg = average;
SD = standard deviation;
RSD = relative standard deviation

Table 21, below, provides individual and average dissolution results of G023 Batch 4209 measured with the proposed G023 method (Paddle 75 rpm).

TABLE 21

| Time | Amount Dissolved (%) | | | | | | | | | | | | | | |
|---|---|---|---|---|---|---|---|---|---|---|---|---|---|---|---|
| (min) | V1 | V2 | V3 | V4 | V5 | V6 | V7 | V8 | V9 | V10 | V11 | V12 | Avg | SD | RSD (%) |
| 5 | 39 | 37 | 43 | 41 | 41 | 43 | 27 | 34 | 43 | 33 | 37 | 39 | 38 | 4.85 | 12.77 |
| 10 | 75 | 70 | 77 | 75 | 76 | 77 | 58 | 70 | 76 | 69 | 72 | 72 | 72 | 5.33 | 7.40 |
| 15 | 93 | 92 | 93 | 91 | 93 | 95 | 83 | 93 | 92 | 90 | 88 | 89 | 91 | 3.19 | 3.51 |
| 20 | 98 | 97 | 97 | 96 | 98 | 100 | 95 | 99 | 97 | 96 | 94 | 95 | 97 | 1.75 | 1.80 |
| 30 | 100 | 99 | 98 | 98 | 99 | 101 | 98 | 101 | 98 | 97 | 95 | 97 | 98 | 1.73 | 1.77 |
| 45 | 100 | 99 | 99 | 98 | 99 | 102 | 99 | 101 | 99 | 98 | 96 | 97 | 99 | 1.62 | 1.64 |
| 60 | 100 | 99 | 99 | 98 | 100 | 102 | 99 | 102 | 99 | 98 | 96 | 98 | 99 | 1.70 | 1.71 |

Avg = average;
SD = standard deviation;
RSD = relative standard deviation

Table 22, below, provides individual and average dissolution results of G023 Batch 4207 stored for 21 months at 30° C./75% RH measured with the proposed G023 Method (Paddle 75 rpm).

TABLE 22

| Time (min) | Vessel 1 | Vessel 2 | Vessel 3 | Vessel 4 | Vessel 5 | Vessel 6 | Avg | SD | RSD (%) |
|---|---|---|---|---|---|---|---|---|---|
| 5 | 41 | 32 | 31 | 25 | 30 | 29 | 32 | 5.36 | 16.99 |
| 10 | 80 | 70 | 63 | 61 | 64 | 63 | 67 | 7.20 | 10.78 |
| 15 | 90 | 84 | 78 | 82 | 80 | 84 | 83 | 3.96 | 4.77 |
| 20 | 93 | 90 | 85 | 90 | 87 | 90 | 89 | 2.66 | 2.99 |
| 30 | 95 | 94 | 91 | 94 | 92 | 94 | 93 | 1.62 | 1.74 |
| 45 | 96 | 95 | 94 | 96 | 94 | 95 | 95 | 1.17 | 1.23 |
| 60 | 97 | 96 | 94 | 97 | 94 | 95 | 96 | 1.19 | 1.24 |

Avg = average;
SD = standard deviation;
RSD = relative standard deviation

Table 23, below, provides dissolution results of G023 Batch 4207 following various storage times and under different temperature conditions.

TABLE 23
Study A15369 - Batch 4207 in HDPE Bottles - Dissolution Results

| Parameter | | | | | | | | |
|---|---|---|---|---|---|---|---|---|
| | Storage | Dissolution Mean (Min-Max) | | | | | | |
| Storage | Time | 5 min | 10 min | 15 min | 20 min | 30 min | 45 min | 60 min |
| Condition | (Months) | (%) | (%) | (%) | (%) | (%) | (%) | (%) |
| 5° C. | Initial | 34 (27-38) | 67 (56-73) | 86 (76-91) | 93 (87-95) | 97 (96-97) | 98 (97-98) | 98 (97-98) |
| | 6 | 28 (25-31) | 63 (58-68) | 85 (79-88) | 94 (92-96) | 97 (95-98) | 98 (96-99) | 98 (96-99) |
| | 12 | 32 (29-38) | 69 (65-71) | 88 (86-89) | 95 (94-96) | 97 (96-98) | 97 (97-99) | 98 (97-99) |
| | 24 | 31 (25-40) | 66 (58-71) | 87 (82-90) | 94 (91-96) | 96 (95-97) | 97 (96-98) | 97 (96-98) |
| | 36 | 34 (30-37) | 69 (66-72) | 88 (87-89) | 94 (92-95) | 96 (95-97) | 97 (96-97) | 97 (96-98) |
| 25° C./60% RH | 3 | 33 (26-38) | 66 (58-70) | 86 (80-89) | 94 (89-95) | 96 (95-97) | 97 (97-98) | 97 (97-98) |
| | 6 | 34 (28-37) | 69 (62-71) | 89 (85-90) | 95 (93-86) | 97 (96-98) | 98 (97-98) | 98 (97-99) |
| | 9 | 29 (25-32) | 64 (58-68) | 85 (82-89) | 93 (92-95) | 97 (96-98) | 98 (97-99) | 98 (97-99) |
| | 12 | 34 (25-43) | 67 (58-74) | 87 (82-90) | 93 (91-94) | 96 (95-96) | 97 (96-97) | 97 (96-97) |
| | 18 | 33 (26-39) | 67 (64-73) | 87 (82-90) | 93 (91-94) | 95 (93-96) | 96 (94-98) | 96 (95-98) |
| | 24 | 36 (31-41) | 70 (65-74) | 88 (85-90) | 93 (91-93) | 95 (94-95) | 95 (95-96) | 96 (95-96) |
| | 36 | 30 (27-34) | 62 (56-68) | 80 (75-84) | 87 (86-89) | 91 (90-92) | 92 (91-93) | 93 (92-93) |
| 30° C./75% RH | 3 | 35 (24-49) | 70 (61-82) | 89 (85-93) | 95 (94-96) | 97 (96-98) | 98 (96-99) | 98 (96-99) |
| | 6 | 34 (29-37) | 70 (64-73) | 90 (87-91) | 95 (94-96) | 97 (96-98) | 98 (96-99) | 98 (97-99) |
| | 9 | 31 (28-36) | 68 (63-71) | 89 (86-91) | 95 (94-96) | 97 (96-98) | 98 (97-99) | 98 (97-99) |
| | 12 | 35 (28-45) | 71 (69-76) | 90 (87-93) | 93 (92-94) | 96 (95-96) | 96 (96-97) | 97 (96-97) |
| | 18 | 33 (28-38) | 68 (66-71) | 87 (86-88) | 92 (91-93) | 94 (93-95) | 95 (94-96) | 96 (95-97) |
| | 24 | 39 (35-42) | 73 (72-74) | 88 (87-88) | 92 (91-92) | 94 (93-95) | 95 (94-96) | 95 (95-96) |
| | 36 | 34 (33-36) | 68 (68-69) | 85 (85-87) | 89 (89-89) | 92 (92-93) | 94 (93-94) | 94 (93-95) |
| 40° C./75% RH | 3 | 32 (24-37) | 69 (61-73) | 89 (86-91) | 94 (94-94) | 96 (96-97) | 96 (96-97) | 97 (96-97) |
| | 6 | 30 (28-36) | 68 (60-72) | 88 (84-90) | 93 (92-94) | 96 (95-97) | 97 (96-95) | 97 (96-98) |
| 50° C. | 3 | 26 (23-29) | 63 (57-69) | 85 (81-89) | 92 (90-94) | 95 (94-96) | 95 (94-98) | 95 (94-96) |
| Light ICH | | 30 (25-35) | 66 (59-70) | 87 (82-89) | 94 (93-95) | 96 (95-97) | 97 (96-98) | 97 (96-98) |

Light ICH = Integrated near UV energy not less than 200 W · h/m², overall illumination not less than 1200 khx h

Table 24, below, provides dissolution results of G023 Batch 4208 following various storage times and under different temperature conditions.

TABLE 24
Study A15370 - Batch 4208 in HDPE Bottles - Dissolution Results

| Parameter | | | | | | | | |
|---|---|---|---|---|---|---|---|---|
| | Storage | Dissolution Mean (Min-Max) | | | | | | |
| Storage | Time | 5 min | 10 min | 15 min | 20 min | 30 min | 45 min | 60 min |
| Condition | (Months) | (%) | (%) | (%) | (%) | (%) | (%) | (%) |
| 5° C. | Initial | 32 (28-37) | 67 (64-70) | 87 (85-89) | 95 (93-96) | 98 (97-99) | 99 (98-100) | 99 (98-100) |
| | 6 | 31 (26-34) | 68 (58-71) | 89 (84-91) | 97 (95-98) | 99 (99-100) | 100 (99-100) | 100 (100-101) |
| | 12 | 33 (27-40) | 67 (59-72) | 86 (77-90) | 94 (89-96) | 97 (95-98) | 97 (96-98) | 98 (97-99) |
| | 24 | 36 (28-40) | 70 (67-73) | 90 (90-90) | 96 (95-97) | 98 (97-99) | 98 (97-99) | 98 (97-99) |
| | 36 | 28 (25-33) | 61 (57-68) | 81 (75-87) | 91 (86-93) | 95 (93-97) | 97 (95-98) | 97 (96-98) |
| 25° C./60% RH | 3 | 35 (28-39) | 69 (62-73) | 90 (87-92) | 97 (96-98) | 99 (98-100) | 100 (99-101) | 100 (99-101) |
| | 6 | 33 (31-35) | 69 (65-72) | 89 (86-91) | 96 (95-97) | 98 (98-99) | 99 (98-100) | 99 (98-100) |
| | 9 | 33 (28-40) | 70 (67-73) | 90 (89-92) | 96 (96-97) | 98 (97-99) | 99 (98-89) | 89 (98-99) |
| | 12 | 29 (25-32) | 63 (55-68) | 85 (80-89) | 93 (91-95) | 97 (95-99) | 98 (97-100) | 98 (96-100) |
| | 18 | 31 (27-35) | 66 (60-70) | 87 (84-89) | 94 (92-95) | 96 (95-97) | 97 (96-98) | 98 (96-98) |
| | 24 | 34 (29-43) | 69 (63-74) | 89 (86-90) | 94 (93-94) | 96 (95-97) | 97 (96-98) | 97 (96-98) |
| | 36 | 31 (23-36) | 65 (56-71) | 86 (81-89) | 92 (91-94) | 95 (94-97) | 97 (95-97) | 97 (96-99) |
| 30° C./75% RH | 3 | 28 (25-33) | 66 (59-70) | 89 (83-81) | 97 (95-98) | 99 (98-101) | 100 (99-101) | 100 (99-101) |
| | 6 | 30 (28-35) | 68 (62-71) | 89 (82-91) | 96 (93-97) | 99 (98-99) | 99 (99-100) | 100 (99-100) |
| | 9 | 33 (27-39) | 69 (64-72) | 90 (88-91) | 95 (94-96) | 97 (96-98) | 98 (97-98) | 98 (97-99) |
| | 12 | 34 (27-41) | 68 (60-74) | 86 (79-91) | 93 (89-95) | 96 (95-97) | 97 (96-98) | 97 (96-98) |
| | 18 | 37 (31-42) | 72 (71-73) | 90 (89-91) | 94 (93-95) | 96 (96-97) | 97 (96-98) | 97 (97-98) |
| | 24 | 35 (30-41) | 71 (68-74) | 88 (85-89) | 92 (90-94) | 95 (94-96) | 96 (95-98) | 96 (95-98) |
| | 36 | 32 (28-36) | 67 (59-71) | 84 (79-85) | 89 (86-90) | 92 (90-93) | 94 (92-95) | 94 (92-95) |
| 40° C./75% RH | 3 | 32 (28-34) | 69 (67-70) | 89 (88-90) | 95 (95-96) | 97 (97-98) | 98 (98-99) | 98 (98-99) |
| | 6 | 33 (27-39) | 70 (64-73) | 89 (87-91) | 94 (93-95) | 96 (96-97) | 97 (97-98) | 98 (97-99) |
| 50° C. | 3 | 28 (24-35) | 63 (57-73) | 86 (82-91) | 94 (93-95) | 96 (95-97) | 96 (95-97) | 96 (95-97) |

Table 25, below, provides dissolution results of G023 Batch 4209 following various storage times and under different temperature conditions.

TABLE 25
Study A15371 - Batch 4209 in HDPE Bottles - Dissolution Results

| Parameter | | | | | | | | |
|---|---|---|---|---|---|---|---|---|
| | Storage | Dissolution Mean (Min-Max) | | | | | | |
| Storage | Time | 5 min | 10 min | 15 min | 20 min | 30 min | 45 min | 60 min |
| Condition | (Months) | (%) | (%) | (%) | (%) | (%) | (%) | (%) |
| 5° C. | Initial | 43 (32-49) | 76 (68-81) | 91 (84-94) | 95 (93-96) | 97 (95-98) | 97 (96-98) | 97 (96-98) |
| | 6 | 37 (30-45) | 73 (67-79) | 91 (89-93) | 96 (94-97) | 97 (95-99) | 98 (95-99) | 98 (95-99) |
| | 12 | 37 (30-43) | 71 (61-78) | 89 (82-92) | 95 (91-98) | 96 (94-100) | 96 (95-100) | 97 (95-100) |
| | 24 | 41 (39-43) | 75 (73-76) | 91 (90-92) | 95 (94-96) | 96 (95-97) | 96 (95-97) | 96 (95-100) |
| | 36 | 40 (32-43) | 74 (70-76) | 91 (89-93) | 95 (94-97) | 96 (95-98) | 97 (96-99) | 97 (96-99) |
| 25° C./60% RH | 3 | 42 (37-44) | 76 (72-81) | 91 (89-93) | 96 (94-98) | 97 (96-100) | 98 (96-100) | 98 (96-100) |
| | 6 | 37 (28-40) | 74 (68-77) | 91 (88-93) | 95 (93-97) | 97 (94-98) | 97 (95-99) | 97 (95-99) |
| | 9 | 40 (36-46) | 75 (71-79) | 92 (90-93) | 95 (93-96) | 96 (94-98) | 97 (95-98) | 97 (95-99) |
| | 12 | 44 (35-49) | 78 (70-82) | 92 (90-93) | 95 (94-96) | 96 (95-96) | 96 (95-97) | 96 (95-97) |
| | 18 | 44 (42-46) | 76 (73-78) | 91 (90-92) | 94 (93-95) | 95 (94-96) | 96 (94-96) | 96 (95-97) |
| | 24 | 46 (45-48) | 79 (77-80) | 91 (90-92) | 93 (92-94) | 95 (93-96) | 95 (94-96) | 95 (94-97) |
| | 36 | 37 (33-43) | 71 (67-76) | 86 (84-89) | 91 (89-94) | 93 (92-97) | 95 (93-97) | 95 (93-98) |
| 30° C./75% RH | 3 | 41 (35-47) | 77 (68-82) | 93 (89-95) | 97 (96-98) | 98 (97-99) | 99 (98-99) | 99 (98-100) |
| | 6 | 41 (39-46) | 77 (74-79) | 92 (91-93) | 95 (93-96) | 97 (94-98) | 97 (95-99) | 97 (95-99) |
| | 9 | 42 (33-47) | 76 (67-80) | 91 (86-93) | 95 (92-96) | 96 (94-97) | 97 (95-98) | 97 (95-98) |
| | 12 | 44 (42-46) | 79 (76-81) | 93 (92-94) | 95 (94-97) | 97 (96-98) | 98 (96-99) | 98 (96-99) |
| | 18 | 43 (40-45) | 76 (74-78) | 90 (88-91) | 93 (91-94) | 94 (92-95) | 95 (92-96) | 95 (93-96) |
| | 24 | 46 (43-51) | 79 (77-81) | 90 (88-91) | 92 (90-93) | 94 (93-96) | 95 (93-97) | 95 (93-97) |
| | 36 | 44 (42-46) | 77 (75-79) | 87 (86-89) | 90 (88-92) | 92 (91-94) | 93 (92-95) | 94 (92-96) |
| 40° C./75% RH | 3 | 42 (29-48) | 74 (56-80) | 88 (75-92) | 93 (90-95) | 95 (93-96) | 95 (94-97) | 96 (95-97) |
| | 6 | 41 (36-45) | 76 (74-78) | 91 (89-93) | 94 (92-96) | 95 (94-98) | 96 (94-99) | 96 (94-99) |
| 50° C. | 3 | 30 (24-38) | 66 (60-76) | 86 (79-92) | 93 (89-95) | 95 (93-96) | 95 (93-97) | 95 (94-97) |

What is claimed:

1. A solid oral dosage form comprising about 500 mg of abiratirone acetate and having a dissolution profile characterized by one or more of features (a)-(f):
(a) about 43% of said dosage form dissolves after five minutes;
(b) about 71% of said dosage form dissolves after 10 minutes;
(c) about 88% of said dosage form dissolves after 20 minutes;
(d) about 94% of said dosage form dissolves after 30 minutes;
(e) about 98% of said dosage form dissolves after 45 minutes; and,
(f) about 99% of said dosage form dissolves after 60 minutes,
when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

2. A solid oral dosage form comprising about 500 mg of abiratirone acetate and having a dissolution profile characterized by one or more of features (a)-(f):
(a) about 31% of said dosage form dissolves after five minutes;
(b) about 65% of said dosage form dissolves after 10 minutes;
(c) about 94% of said dosage form dissolves after 20 minutes;
(d) about 98% of said dosage form dissolves after 30 minutes;
(e) about 99% of said dosage form dissolves after 45 minutes; and,
(f) about 99% of said dosage form dissolves after 60 minutes,
when measured by the USP 2 Paddle method at 75 rpm in 900 mL of an aqueous solution comprising 56.5 mM phosphate buffer with 0.25% (w/v) sodium lauryl sulfate at pH 4.5 and a temperature of 37.0±0.5° C.

3. A solid oral dosage form comprising:
about 500 mg of abiraterone acetate; and
a film coating that is positioned on an outer surface of said dosage form,
wherein said dosage forms are bioequivalent, when administered orally on an equivalent dose basis, to 250 mg ZYTIGA® abiraterone acetate dosage forms.

4. The dosage form according to claim 1 comprising about 30 to about 50 wt % of said abiraterone acetate.

5. The dosage form according to claim 4 comprising about 35 to about 45 wt % of said abiraterone acetate.

6. The dosage form according to claim 5 comprising about 35 wt % of said abiraterone acetate.

7. The dosage form according to claim 5 comprising about 45 wt % of said abiraterone acetate.

8. The dosage form according to claim 1 further comprising one or more of a diluent, a disintegrant, a binder, a surfactant, a glidant, or a lubricant.

9. The dosage form according to claim 2 further comprising a diluent, a disintegrant, a binder, a surfactant, a glidant, and a lubricant.

10. The dosage form according to claim 8 further comprising a diluent that is selected from lactose monohydrate, microcrystalline cellulose, and silicified microcrystalline cellulose.

11. The dosage form according to claim 8 further comprising a disintegrant, wherein said disintegrant is croscarmellose sodium.

12. The dosage form according to claim 8 further comprising a binder that is selected from povidone and hypromellose.

13. The dosage form according to claim 8 further comprising a surfactant, wherein said surfactant is sodium lauryl sulfate.

14. The dosage form according to claim 8 comprising lactose monohydrate, microcrystalline cellulose or silicified microcrystalline cellulose, croscarmellose sodium, povidone or hypromellose, sodium lauryl sulfate, colloidal silicon dioxide, and magnesium stearate.

15. The dosage form according to claim 14 comprising about 22-28 wt % lactose monohydrate, about 16-17 wt % microcrystalline cellulose or about 18-20 wt % silicified microcrystalline cellulose, about 6-7 wt % croscarmellose sodium, about 4-6 wt % povidone or about 1-2 wt % hypromellose, about 4-6 wt % sodium lauryl sulfate, about 0.5-1.5 wt % colloidal silicon dioxide, and about 1-2 wt % magnesium stearate.

16. The dosage form according to claim 15, wherein said dosage form comprises about 35 wt % abiraterone acetate, about 28 wt % lactose monohydrate, about 20 wt % silicified microcrystalline cellulose, about 6 wt % croscarmellose sodium, about 5 wt % povidone, about 1 wt % colloidal silicon dioxide, and about 1.5 wt % magnesium stearate, wherein the combination of each of said components in the dosage form equals 100 wt %.

17. The dosage form according to claim 15, wherein said dosage form comprises about 45 wt % abiraterone acetate, about 23 wt % lactose monohydrate, about 17 wt % microcrystalline cellulose, about 6 wt % croscarmellose sodium, about 2 wt % hypromellose, about 1 wt % colloidal silicon dioxide, and about 1.5 wt % magnesium stearate, wherein the combination of each of said components in the dosage form equals 100 wt %.

18. The dosage form according to claim 1, wherein two of said dosage forms administered orally at substantially the same time exhibit a Cₘₐₓ of about 130 ng/mL.

19. The dosage form according to claim 1, wherein two of said dosage forms administered orally at substantially the same time exhibit a Cₘₐₓ of about 108 ng/mL.

20. The dosage form according to claim 1, wherein two of said dosage forms administered orally at substantially the same time exhibit a tₘₐₓ at about 2.0 hours.

21. The dosage form according to claim 1, wherein two of said dosage forms administered orally at substantially the same time exhibit an area under the plasma concentration-time curve from time zero to the time of the last quantifiable concentration (AUCₗₐₛₜ) of about 679 ng*h/mL.

22. The dosage form according to claim 1, wherein two of said dosage forms administered orally at substantially the same time exhibit an area under the plasma concentration-time curve from time zero to the time of the last quantifiable concentration (AUCₗₐₛₜ) of about 589 ng*h/mL.

23. The dosage form according to claim 1, wherein two of said dosage forms administered orally at substantially the same time exhibit an area under the plasma concentration-time curve extrapolated to infinite time (AUC_{∞}) of about 707 ng*h/mL.

24. The dosage form according to claim 1, wherein two of said dosage forms administered orally at substantially the same time exhibit an area under the plasma concentration-time curve extrapolated to infinite time (AUC_{∞}) of about 600 ng*h/mL.

25. The dosage form according to claim 1, wherein two of said dosage forms administered orally at substantially the same time exhibit an apparent terminal elimination half-life (t_{1/2}) of about 18.6 hours.

26. The dosage form according to claim 1, wherein two of said dosage forms administered orally at substantially the same time exhibit an apparent terminal elimination half-life (t_{1/2}) of about 18.1 hours.

27. A method of reducing pill burden on a subject in need of an abiraterone acetate pharmaceutical regimen comprising orally administering to a subject two dosage forms according to claim 1 at substantially the same time.

28. A method of treating a subject using an abiraterone acetate pharmaceutical regimen that is bioequivalent to 250 mg ZYTIGA® abiraterone acetate dosage forms when administered orally on an equivalent dose basis, comprising orally administering a dosage form according to claim 27.

29. A method of treating a subject who has prostate cancer comprising orally administering to said subject a dosage form according to claim 1.

30. The method according to claim 29 comprising orally administering to said subject two of said dosage forms at substantially the same time.

31. A method of selling a drug product comprising abiraterone acetate, said method comprising selling such drug product, wherein a drug product label for a reference listed drug for such drug product includes instructions for treating non-metastatic castration resistant prostate cancer.

32. The method of claim 31, wherein the drug product is an ANDA drug product, a supplemental New Drug Application drug product or a 505(b)(2) drug product.

33. A method of offering for sale a drug product comprising abiraterone acetate, said method comprising offering for sale such drug product, wherein a drug product label for a reference listed drug for such drug product includes instructions for treating non-metastatic castration resistant prostate cancer.

34. The method of claim 33, wherein the drug product is an ANDA drug product, a supplemental New Drug Application drug product or a 505(b)(2) drug product.

35. A method of selling a drug product comprising abiraterone acetate, said method comprising selling such drug product, wherein the drug product label for a reference listed drug for such drug product comprises metastasis free survival data.

36. The method of claim 35, wherein the metastasis free survival data for abiraterone acetate in combination with androgen deprivation therapy arm has a median of about 40.5 months.

37. A method of offering for sale a drug product comprising abiraterone acetate, said method comprising offering for sale such drug product, wherein the drug product label for a reference listed drug for such drug product comprises metastasis free survival data.

38. The method of claim 37, wherein the metastasis free survival data for abiraterone acetate in combination with androgen deprivation therapy arm has a median of about 40.5 months.

39. An approved drug product comprising 500 mg abiraterone acetate.

40. An approved drug product of claim 39, wherein the approved drug product is a NDA drug product, an ANDA drug product, a supplemental New Drug Application drug product, or a 505(b)(2) drug product.

41. An approved drug product of claim 39, wherein a reference listed drug product for the approved drug product includes a drug product label.

42. An approved drug product of claim 41, wherein the drug product label comprises metastasis free survival data.
